(19) United States
(12) Patent Application Publication
(10) Pub. No.: US 2025/0222222 A1
(43) Pub. Date: Jul. 10, 2025

(54) PATIENT INTERFACE HAVING UNDERCUSHION AND MEMBRANE

(71) Applicant: ResMed Pty Ltd, Bella Vista, New South Wales (AU)

(72) Inventors: Balint FRANKO, Sydney (AU); Rupert Christian SCHEINER, Sydney (AU)

(21) Appl. No.: 18/851,330

(22) PCT Filed: Mar. 30, 2023
(86) PCT No.: PCT/AU2023/050234

§ 371 Date: Sep. 26, 2024

(30) Foreign Application Priority Data
Mar. 30, 2022 (AU) 2022900823

(51) Int. Cl. A61M 16/06

(52) U.S. Cl. CPC A61M 16/0622; A61M 16/0683; A61M 2205/0216

(57) ABSTRACT

A patient interface comprising: a frame partially forming a plenum chamber and comprising a channel around a periphery of the frame; at least one pair of headgear connector portions connected to the frame; a seal-forming structure connected to the frame and partially forming the plenum chamber, wherein the seal forming structure comprises an undercushion and a membrane portion connected to the undercushion and configured to inflate, in use, to form a seal to at least a pronasale region and nasal alae of the patient's nose, the seal forming structure further configured to form a seal around the patient's mouth; and wherein the undercushion of the seal-forming structure comprises a connection portion on a non-patient facing side of the undercushion, the connection portion being configured to be received in the channel of the frame to releasably connect the seal-forming structure to the frame.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched gas at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 1.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

Some patient interfaces of the prior art comprise a cushion module having a rigid shell with a predefined shape based on the anthropometrics of a notional person in the middle of a selected size range. As a result, the seal and comfort success with a given size of cushion module may be strongly related to the correlation between the patient's anthropometrics and those of the notional person on which the design is based. This may result in the need to “fit” the patient (possibly with assistance from a suitably qualified professional) to a particular size of cushion module and/or a particular type of interface. This may also result in the need to manufacture a range of different size cushion modules to ensure that a broad range of patients can find a size with suits them.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material, for example silicone, positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

Many of the seal forming structures of the prior art comprise an element made from silicone (or another similar polymer) which creates a seal against the patient's face. However, some patients may dislike the surface texture of silicone and/or its lack of breathability.

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition in cooler climates, warm air applied generally to the face area in and about the patient interface is more comfortable than cold air. Humidifiers therefore often have the capacity to heat the flow of air was well as humidifying it.

### 1.2.3.5 Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched gas are condensed. POCs seek to utilize their produced oxygen as efficiently as possible, in order to minimise weight, size, and power consumption. This may be achieved by delivering the oxygen as series of pulses or “boli”, each bolus timed to coincide with the start of inspiration. This therapy mode is known as pulsed or demand (oxygen) delivery (POD), in contrast with traditional continuous flow delivery more suited to stationary oxygen concentrators.

### 1.2.3.6 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 1.2.3.7 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focussed airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

TABLE of noise of prior masks (ISO 17510-2:
2007, 10 cmH₂O pressure at 1 m)

| | | A-weighted | A-weighted | |
|---|---|---|---|---|
| | | sound power | sound pressure | |
| | Mask | level dB(A) | dB(A) | Year |
| Mask name | type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| Mirage ™ (*) | | | | |
| ResMed | nasal | 36 (3) | 28 (3) | 2000 |
| UltraMirage ™ | | | | |
| ResMed Mirage | nasal | 32 (3) | 24 (3) | 2002 |
| Activa ™ | | | | |
| ResMed Mirage | nasal | 30 (3) | 22 (3) | 2008 |
| Micro ™ | | | | |
| ResMed Mirage ™ | nasal | 29 (3) | 22 (3) | 2008 |
| SoftGel | | | | |
| ResMed Mirage ™ | nasal | 26 (3) | 18 (3) | 2010 |
| FX | | | | |
| ResMed Mirage | nasal | 37 | 29 | 2004 |
| Swift ™ (*) | pillows | | | |
| ResMed Mirage | nasal | 28 (3) | 20 (3) | 2005 |
| Swift ™ II | pillows | | | |
| ResMed Mirage | nasal | 25 (3) | 17 (3) | 2008 |
| Swift ™ LT | pillows | | | |
| ResMed AirFit | nasal | 21 (3) | 13 (3) | 2014 |
| P10 | pillows | | | |

(*one specimen only, measured using test method specified in ISO 3744 in CPAP mode at 10 cmH₂O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at 1 m |
| Walter Broadly Litter Hog: B+ | | distance |
| Grade | | |
| Conversational speech | 60 | 1 m distance |
| Average home | 50 | |
| Quiet library | 40 | |
| Quiet bedroom at night | 30 | |
| Background in TV studio | 20 | |

### 1.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

Another aspect of one form of the present technology is to provide a patient interface that can flex to accommodate patients having faces of varying widths.

Another aspect of one form of the present technology is to provide an oro-nasal patient interface that is lightweight.

Another aspect of one form of the present technology is to provide an oro-nasal patient interface that is comfortable and low cost.

Another form of the present technology comprises a patient interface comprising:
a frame formed from a flexible material and partially forming a plenum chamber pressurisable to a therapeutic pressure of at least 6 cmH2O above ambient air pressure, the frame comprising a channel around a periphery of the frame, the plenum chamber having at least one plenum chamber inlet port sized and structured to receive a flow of air at the therapeutic pressure for breathing by the patient;
at least one pair of headgear connection portions connected to the frame;
a seal-forming structure releasably connectable to the frame and partially forming the plenum chamber, the seal-forming structure being constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
wherein the seal forming structure comprises a foam undercushion and a membrane portion connected to the foam undercushion and configured to inflate, in use, to form a seal to at least a pronasale region and nasal alae of the patient's nose, the seal forming structure further configured to form a seal around the patient's mouth;
wherein the foam undercushion of the seal-forming structure comprises a connection portion on a non-patient facing side of the foam undercushion, the connection portion being configured to be received in the channel of the frame to releasably connect the seal-forming structure to the frame.

In examples:
the frame is formed from an elastomeric material;
the frame is formed from silicone;
the silicone forming the frame has a 40 Shore A Durometer hardness;
the channel extends around an entire periphery of the frame;
the channel and the foam undercushion engage to form a seal;
the foam undercushion forms a male portion configured to engage with a female portion of the frame;
the channel has a substantially C-shaped cross-section;
the channel opens outwardly with respect to the frame and the connection portion of the foam undercushion comprises an opening having a periphery facing inwardly with respect to the opening, the periphery of the opening being configured to fit within the channel;
the connection portion is configured to be press fit into the channel;
the foam undercushion is formed from a polyurethane, for example a thermoplastic polyurethane;
the foam undercushion is formed from a thermoplastic elastomer;
the foam undercushion is configured to hold the membrane portion substantially taut when the patient interface is not in use;
the membrane portion is attached to the foam undercushion around an outer periphery of the membrane portion;
the membrane portion is bonded to the foam undercushion;
the membrane portion comprises a first hole through which air can flow to both the patient's nares, in use;
the membrane portion is configured to be stretched by the patient's nose proximate the first hole in use;
the membrane portion comprises a second hole through which air can flow to the patient's mouth, in use;
the membrane portion is at least partially formed from a textile material;
the textile material forms a patient-facing side of the membrane portion and the membrane portion further comprises an air impermeable coating on a non-patient facing side thereof;
the air impermeable coating comprises a silicone layer;
the silicone layer has a thickness within the range of 0.02 mm-0.05 mm;
the total thickness of the membrane portion is substantially 0.3 mm; and/or
the membrane portion is formed from a single sheet.

In further examples:
the foam undercushion comprises a nasal portion configured to be positioned proximate an inferior periphery of the patient's nose in use, the foam undercushion comprising a nasal recess in the nasal portion configured to prevent the seal-forming structure from occluding the patient's nose;
the nasal recess comprises a shape corresponding to the inferior periphery of the patient's nose; and/or
the membrane portion is attached to the foam undercushion around the periphery of the nasal recess.

In further examples:
the frame has a non-zero negative first principal curvature and a second principal curvature which is less than the first principal curvature;
the second principal curvature is substantially zero and substantially parallel, in use, to the patient's sagittal plane;
the frame flexes such that the first principal curvature has a larger magnitude when donned by a patient with a narrow face than when donned by a patient having a relatively wider face;
the at least one pair of headgear connector portions comprises a pair of superior headgear connector portions connected to the frame and a pair of inferior headgear connector portions connected to the frame;
each of the superior headgear connector portions comprises a curved arm; and/or
each of the inferior headgear connector portions comprises a magnetic connector.

Another form of the present technology comprises a patient interface comprising:
a frame partially forming a plenum chamber pressurisable to a therapeutic pressure of at least 6 cmH2O above ambient air pressure, the plenum chamber having at least one plenum chamber inlet port sized and structured to receive the flow of air at the therapeutic pressure for breathing by the patient;
at least one pair of headgear connector portions connected to the frame;
a seal-forming structure connected to the frame and partially forming the plenum chamber, the seal-forming structure being constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
wherein the seal forming structure comprises an undercushion and a membrane portion connected to the undercushion and configured to inflate, in use, to form a seal to at least a pronasale region and the nasal alae of the patient's nose, the seal forming structure further configured to form a seal around the patient's mouth;
wherein the undercushion comprises a nasal portion configured to be positioned proximate an inferior periphery of the patient's nose in use, the undercushion comprising a nasal recess in the nasal portion comprising an inwardly-facing wall structured to face and at least partially surround the inferior periphery of the patient's nose in use, the membrane portion being supported by the undercushion at a periphery of the nasal recess.

In examples:
the nasal recess is configured to avoid engaging both lateral sides of the patient's nose simultaneously;
the nasal recess is structured to provide substantially no medially-directed forces on the patient's nasal alae in use;
the nasal recess is structured and arranged such that the undercushion applies substantially no force on the patient's nose in use;
the nasal recess is configured to prevent the seal-forming structure from occluding the patient's nose in use;
the nasal recess comprises a shape corresponding to the inferior periphery of the patient's nose;
the nasal recess surrounds substantially all laterally- and anteriorly-facing portions of the inferior periphery of the patient's nose in use;
the inwardly-facing wall extends from at or proximate one of the patient's cheeks around the patient's nose to at or proximate the other of the patient's cheeks;
the membrane portion is attached to the undercushion around the periphery of the nasal recess;
the undercushion comprises a superiorly-facing surface located adjacent to and provided around the periphery of the nasal recess, the membrane portion being attached to the superiorly-facing surface of the undercushion;
the inwardly-facing wall has a concave cross-section in a horizontal plane parallel with the Frankfort horizontal plane of the patient's head;
the inwardly-facing wall has a concave cross-section in the sagittal plane and/or in a vertical plane parallel to the coronal plane;
the undercushion comprises a pair of posterior support portions each positioned on a respective lateral side of the nasal recess and configured to engage the patient's face medially of and proximate to the nasolabial sulci of the patient's face;
the posterior support portions engage the patient's face at locations inferior to the nasal alae on either lateral side of the patient's lip superior and/or at locations aligned vertically with the patient's nasal alae between the nasal alae and the nasolabial sulci;
the posterior support portions are shaped to protrude at least partially medially into concavities formed on the patient's face on either lateral side of the patient's nasal alae; and/or
the inwardly-facing wall extend from a first one of the posterior support portions around the patient's nose to the other one of the posterior support portions.

In further examples:
the seal-forming structure is releasably connected to the frame;
the frame comprises a channel around a periphery of the frame and the undercushion of the seal-forming structure comprises a connection portion on a non-patient facing side of the undercushion, the connection portion being configured to be received in the channel of the frame to releasably connect the seal-forming structure to the frame;
the channel extends around an entire periphery of the frame;
the channel and the undercushion engage to form a seal;
the undercushion forms a male portion configured to engage with a female portion of the frame;
the channel has a substantially C-shaped cross-section;
the channel opens outwardly with respect to the frame and the connection portion of the undercushion comprises an opening having a periphery facing inwardly with respect to the opening, the periphery of the opening being configured to fit within the channel;
the connection portion is configured to be press fit into the channel;
the undercushion is configured to hold the membrane portion substantially taut when the patient interface is not in use;
the membrane portion is attached to the undercushion around an outer periphery of the membrane portion;
the membrane portion is bonded to the undercushion;
the membrane portion comprises a first hole through which air can flow to both the patient's nares, in use;
the membrane portion is configured to be stretched by the patient's nose proximate the first hole in use;
the membrane portion comprises a second hole through which air can flow to the patient's mouth, in use;
the membrane portion is at least partially formed from a textile material;
the textile material forms a patient-facing side of the membrane portion and the membrane portion further comprises an air impermeable coating on a non-patient facing side thereof;
the air impermeable coating comprises a silicone layer;
the silicone layer has a thickness within the range of 0.02 mm-0.05 mm;
the total thickness of the membrane portion is substantially 0.3 mm; and/or
the membrane portion is formed from a single sheet.
the undercushion is formed from foam;
the foam undercushion is formed from a polyurethane, for example a thermoplastic polyurethane;
the foam undercushion is formed from a thermoplastic elastomer;
the frame is formed from a flexible material;
the frame is formed from silicone;
the silicone forming the frame has a 40 Shore A Durometer hardness;
the channel extends around an entire periphery of the frame;
the frame has a non-zero negative first principal curvature and a second principal curvature which is less than the first principal curvature;
the second principal curvature is substantially zero and substantially parallel, in use, to the patient's sagittal plane;
the frame flexes such that the first principal curvature has a larger magnitude when donned by a patient with a narrow face than when donned by a patient having a relatively wider face;
the at least one pair of headgear connectors comprises a pair of superior headgear connectors connected to the frame and a pair of inferior headgear connectors connected to the frame;
each of the superior headgear connectors comprises a curved arm; and/or
each of the inferior headgear connectors comprises a magnetic connector.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

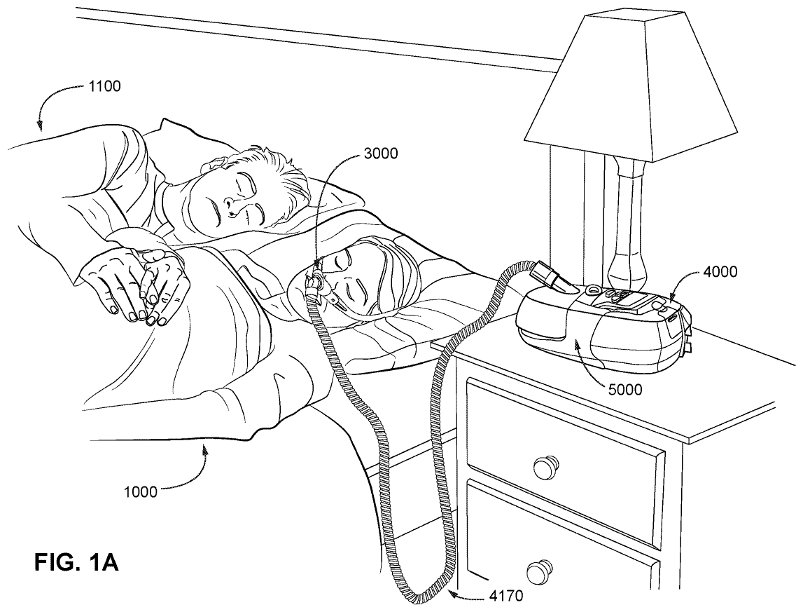

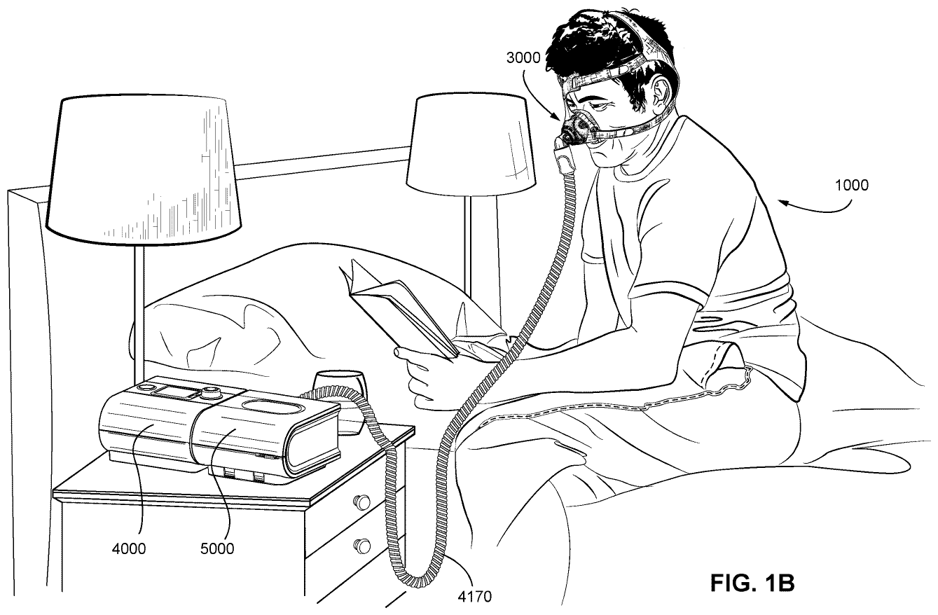

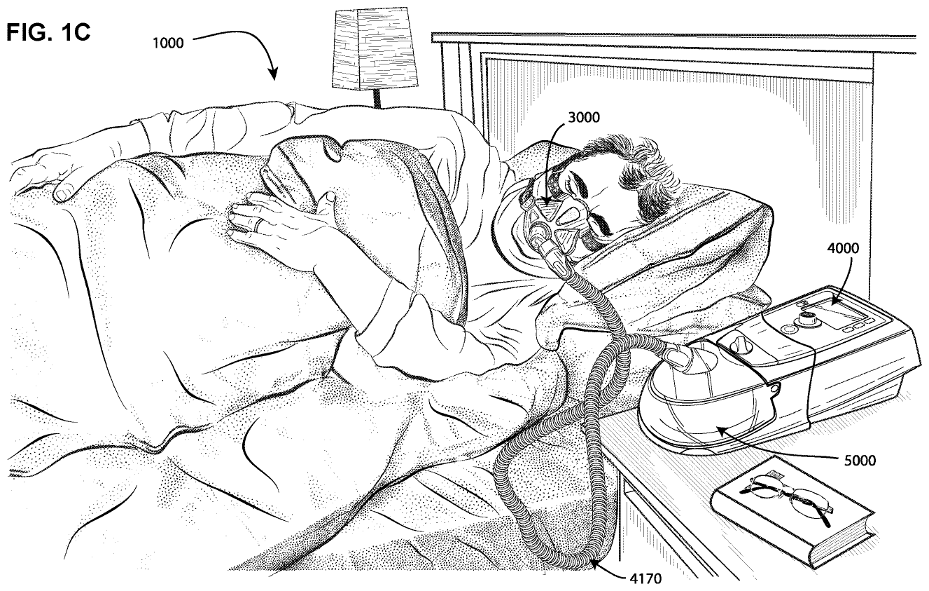

FIG. 1A shows a system including a patient 1000 wearing a patient interface 3000, in the form of nasal pillows, receiving a supply of air at positive pressure from an RPT device 4000. Air from the RPT device 4000 is conditioned in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

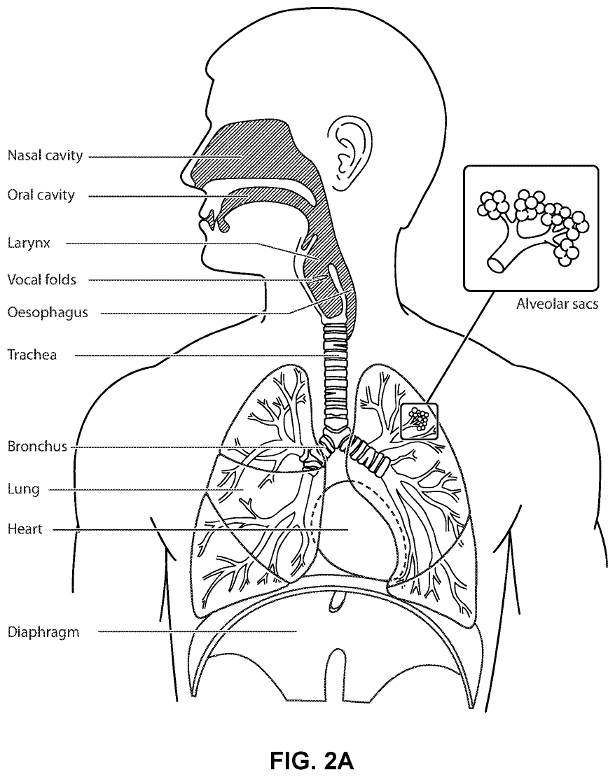

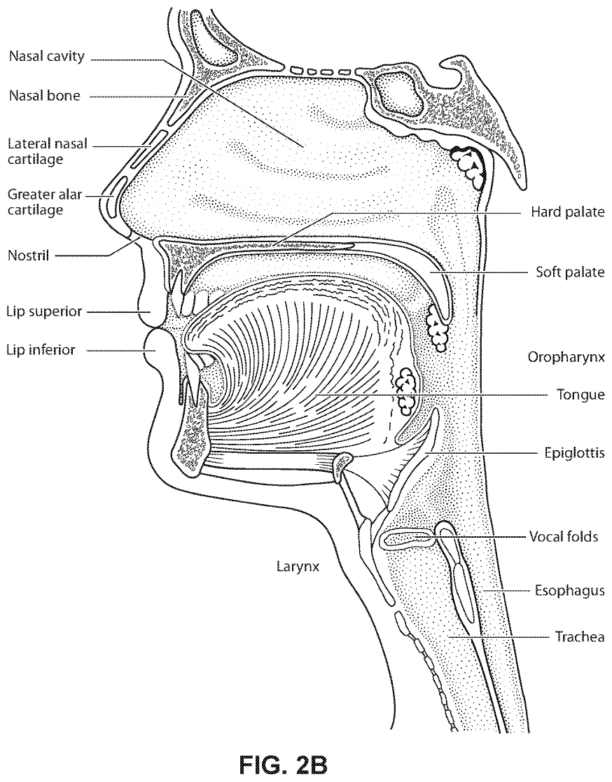

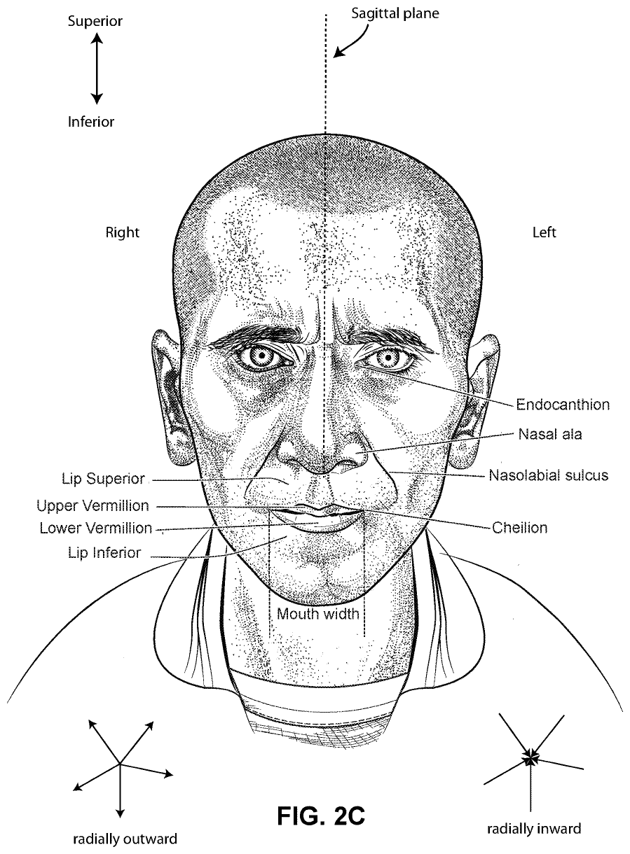

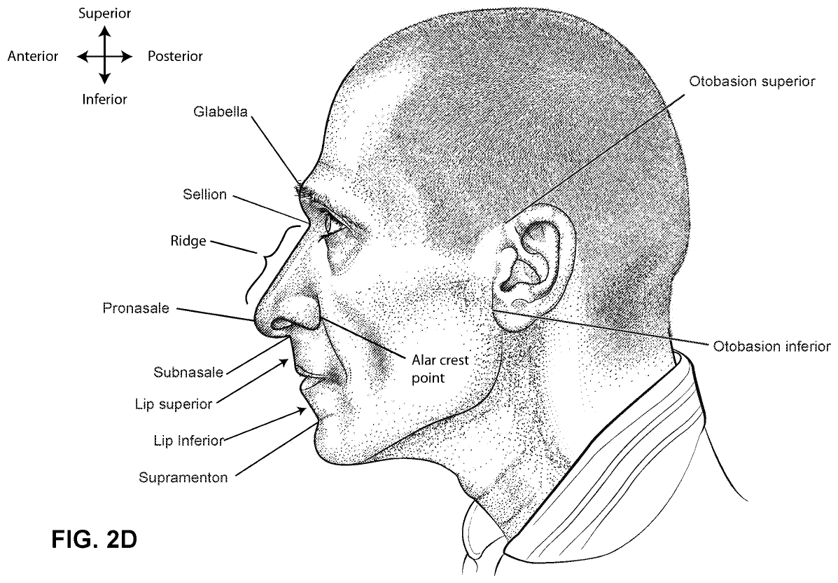

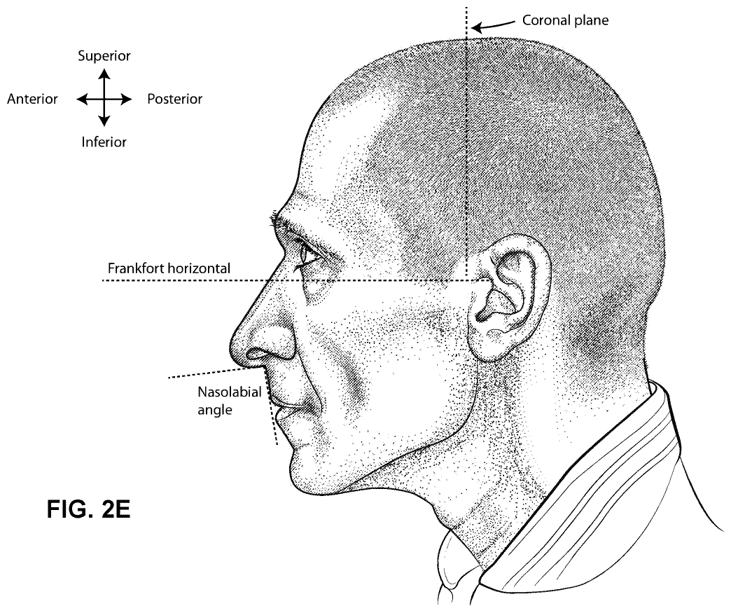

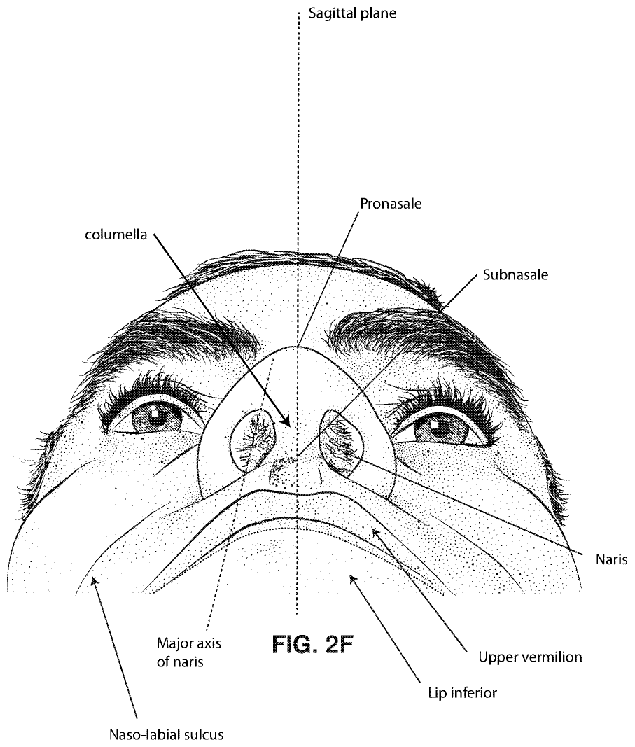

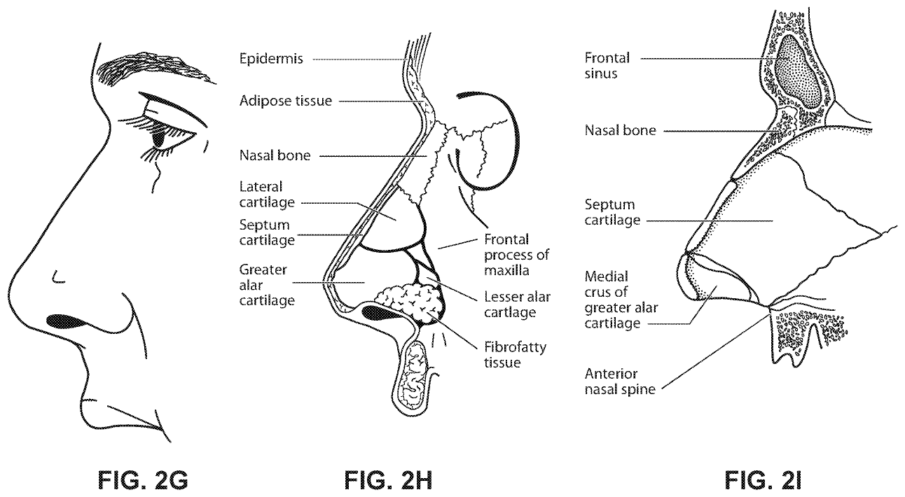

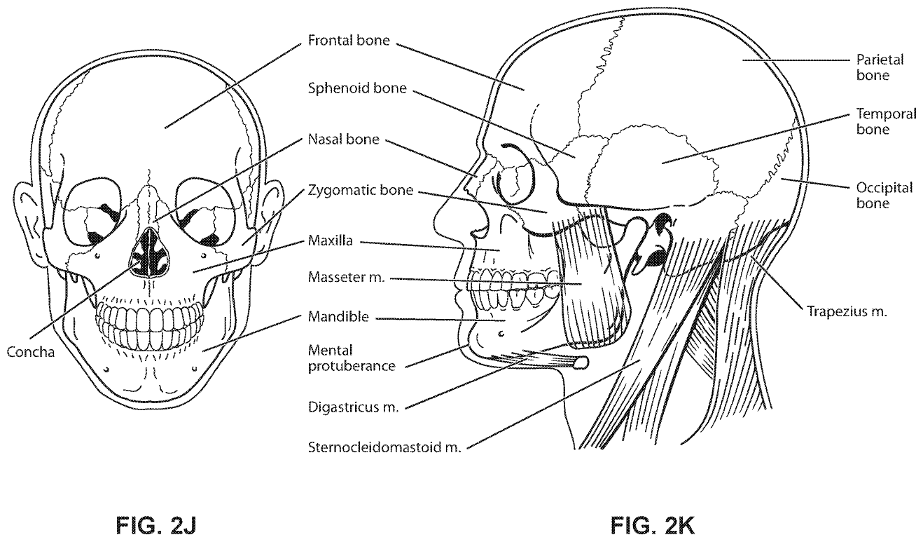

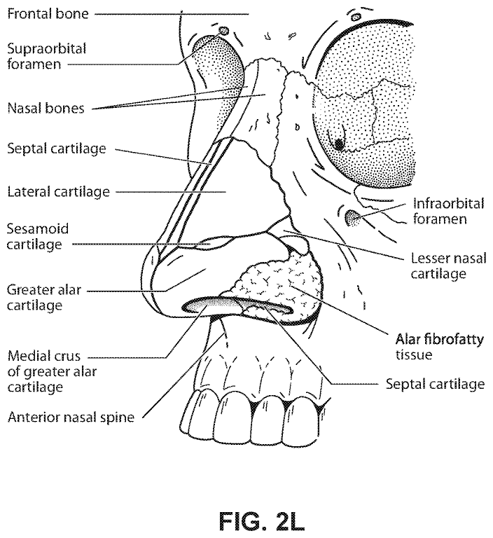

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideotrapezius.

FIG. 2L shows an anterolateral view of a nose.

### 3.3 Patient Interface

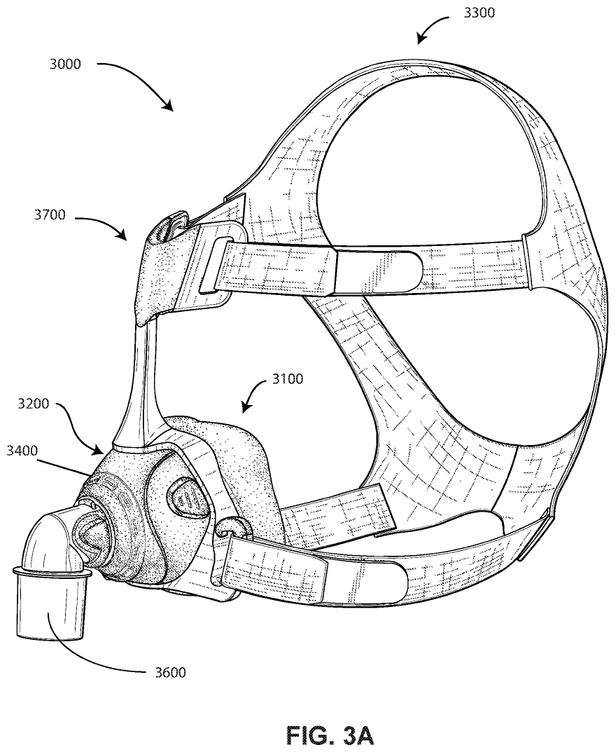

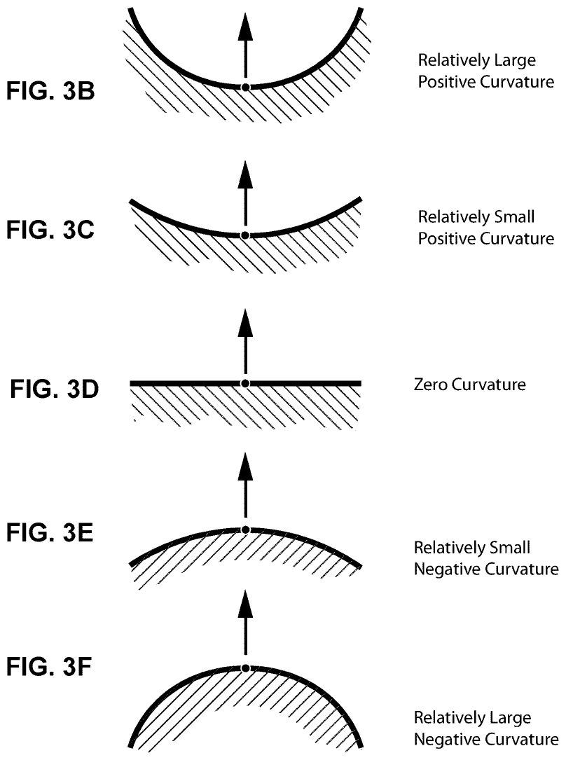

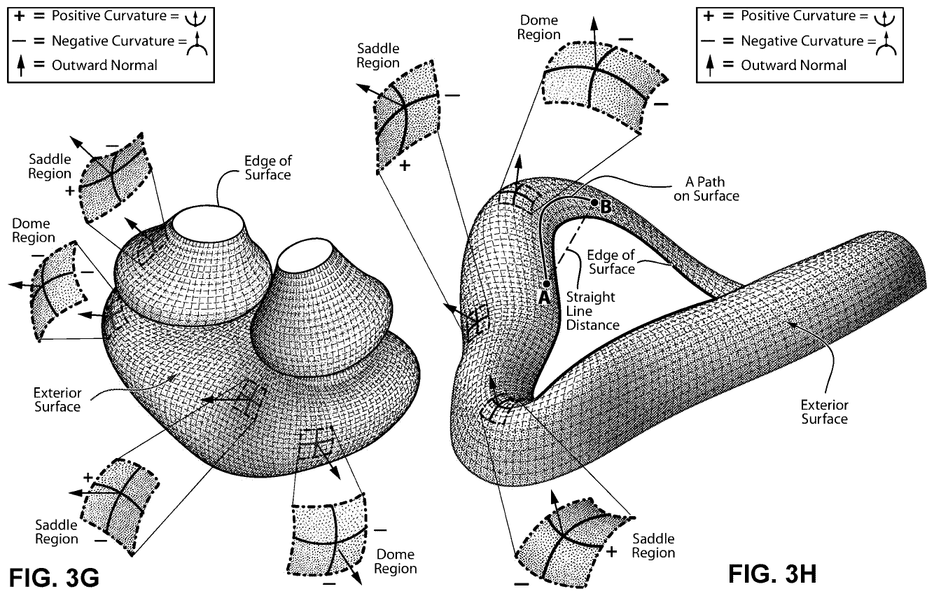

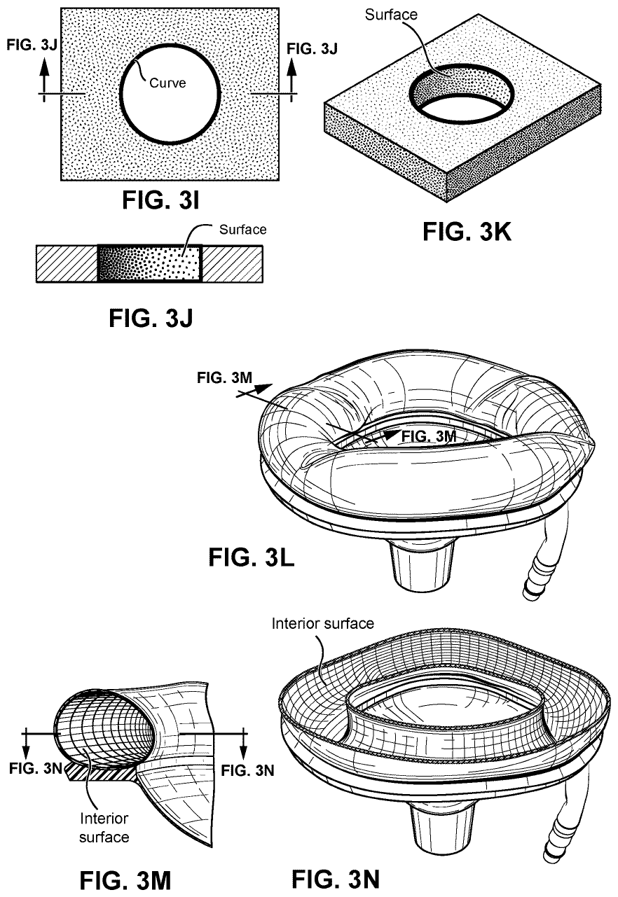

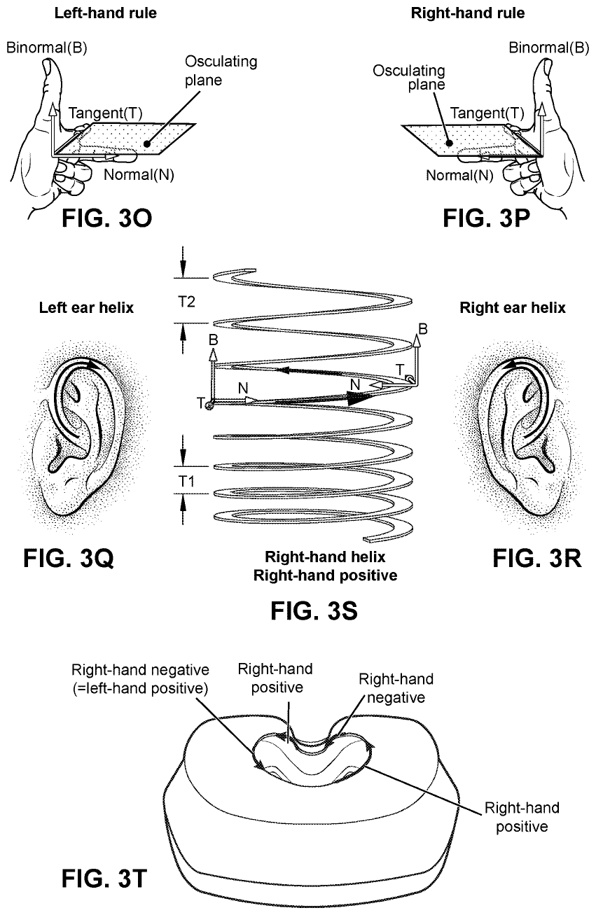

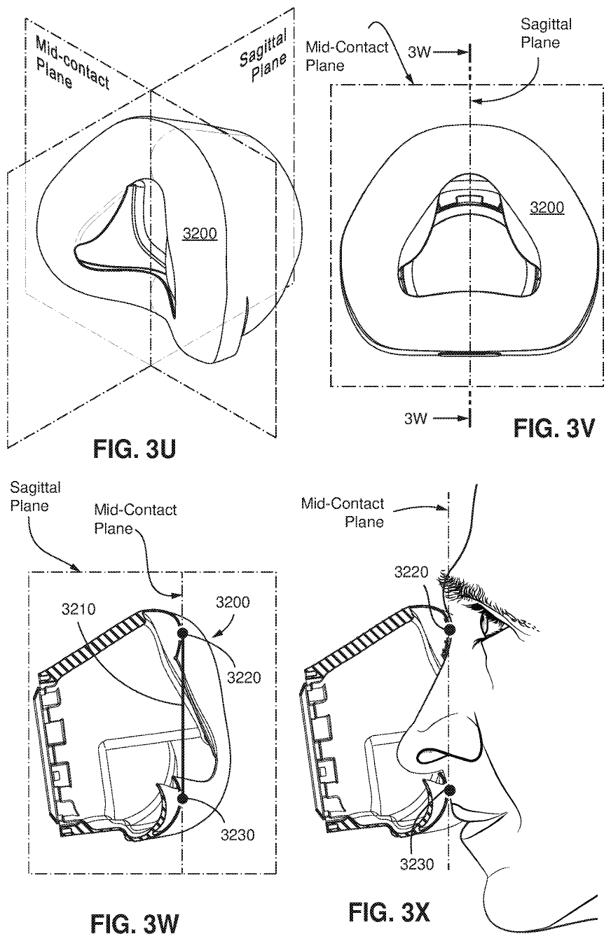

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

FIG. 3Y shows a patient interface in the form of a nasal cannula in accordance with one form of the present technology.

### 3.4 RPT Device

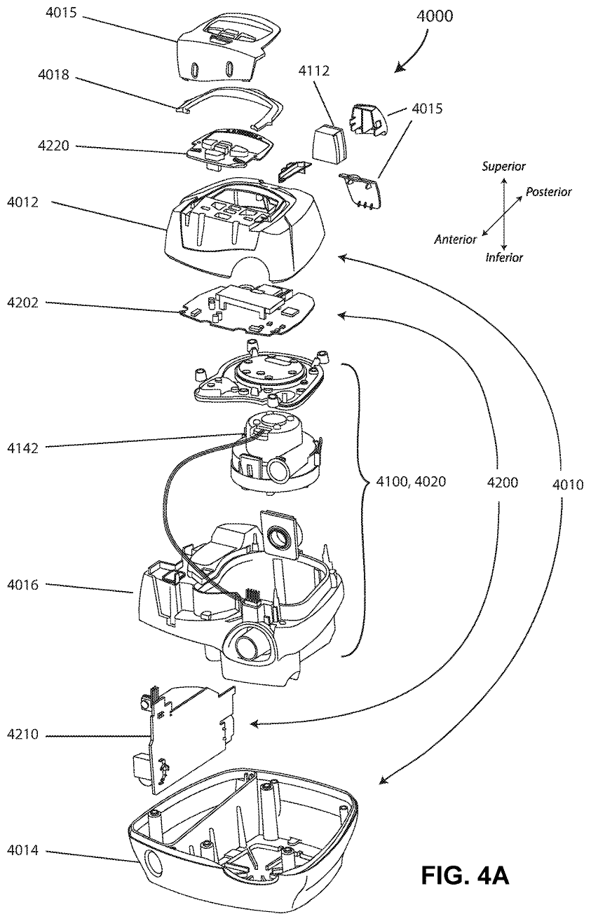

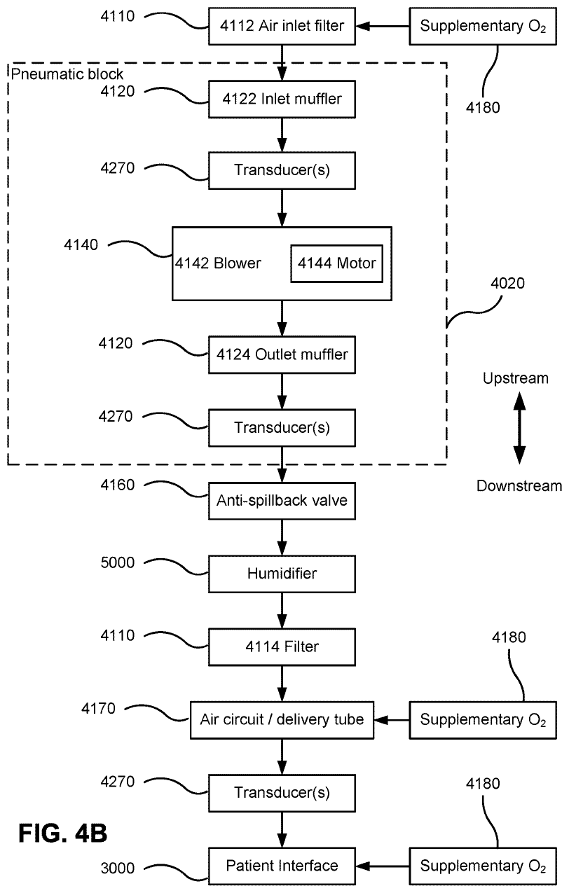

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 Humidifier

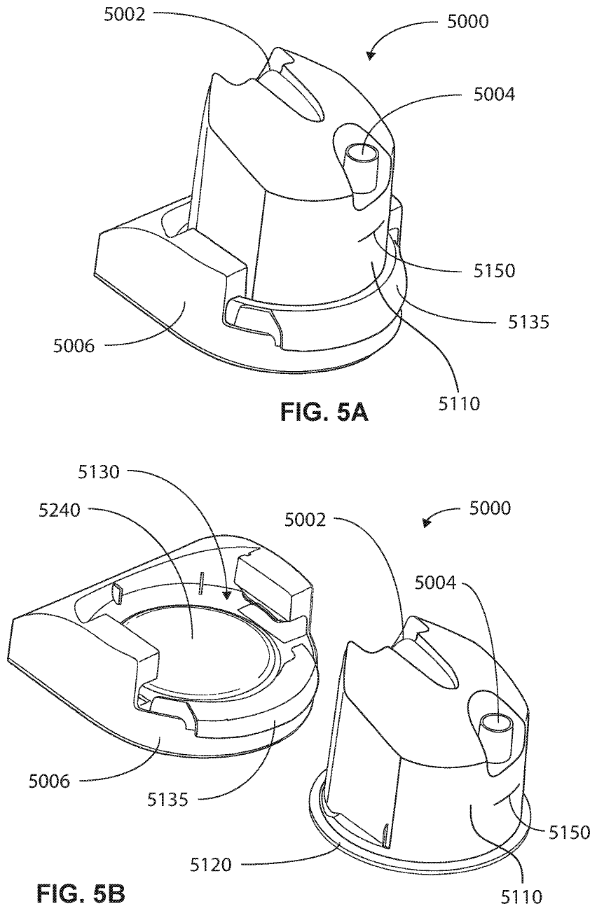

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 Breathing Waveforms

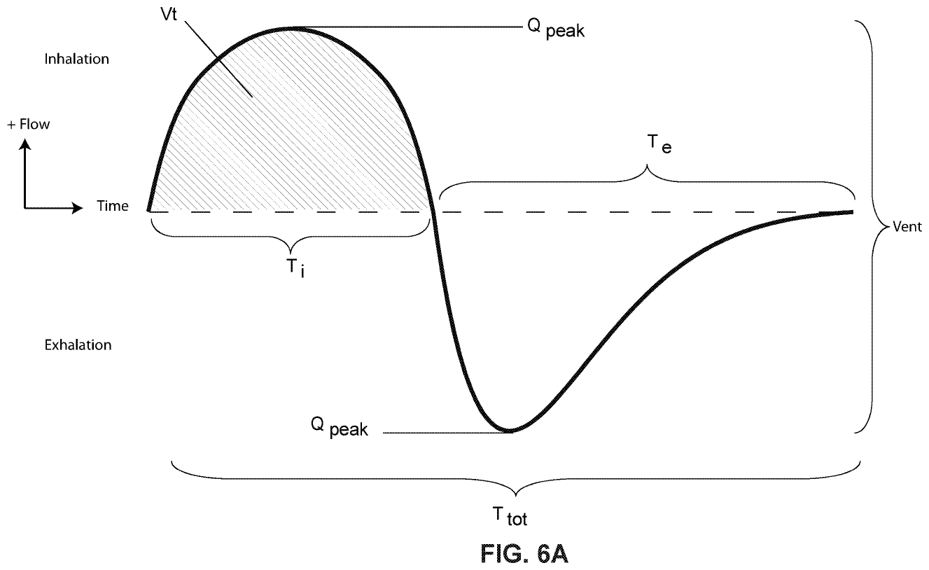

FIG. 6A shows a model typical breath waveform of a person while sleeping.

### 3.7 Patient Interfaces of the Present Technology

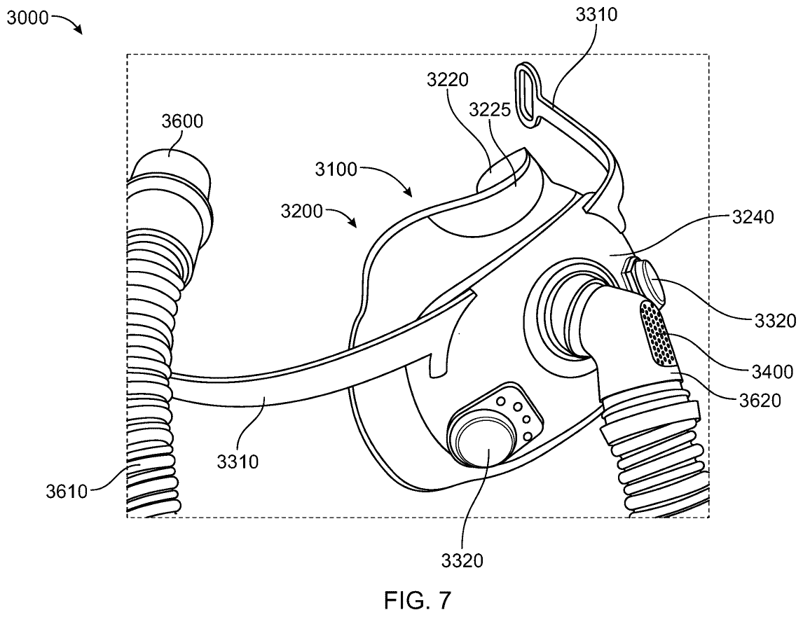

FIG. 7 shows a perspective view of a patent interface according to one form of the technology.

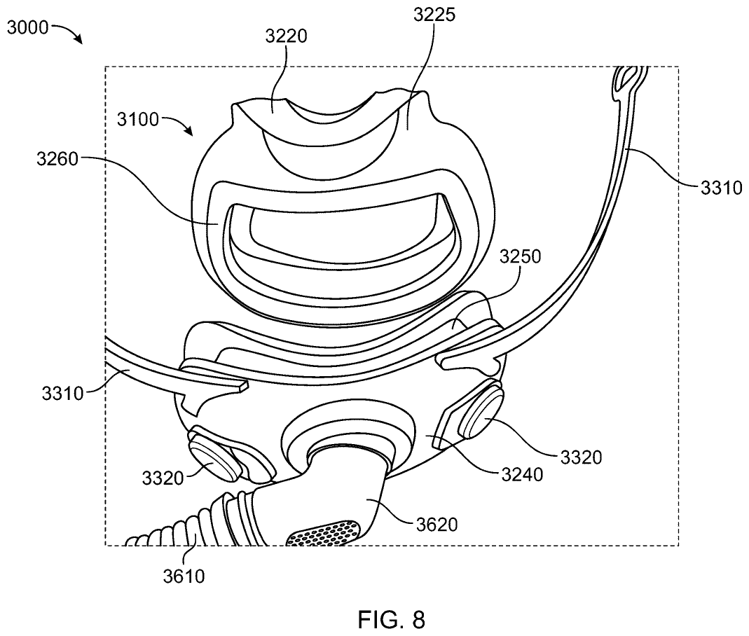

FIG. 8 is an exploded perspective view of the patient interface of FIG. 7.

FIG. 9 is a perspective view of a frame of the patent interface of FIG. 7.

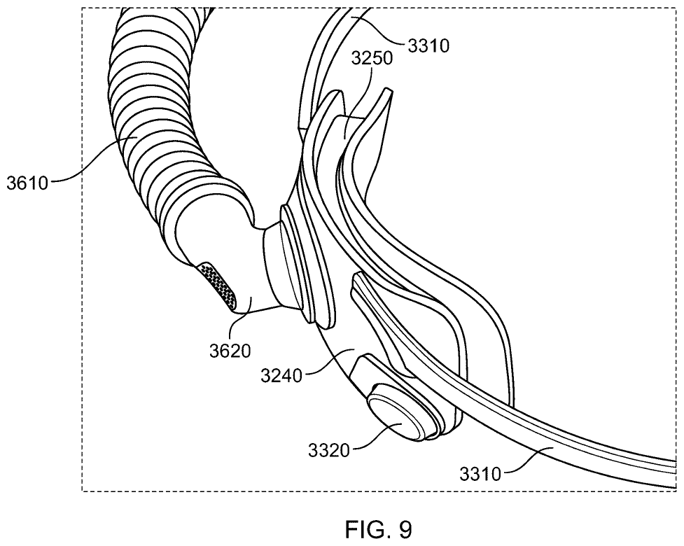

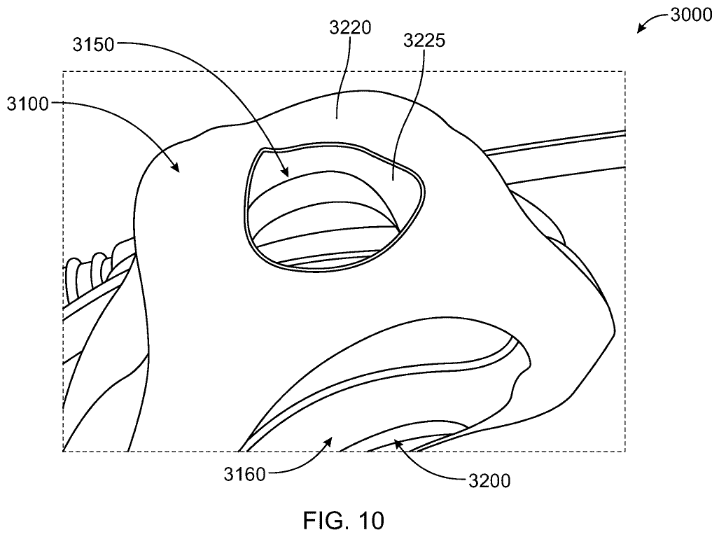

FIG. 10 is a posterosuperior view of the patient interface of FIG. 7.

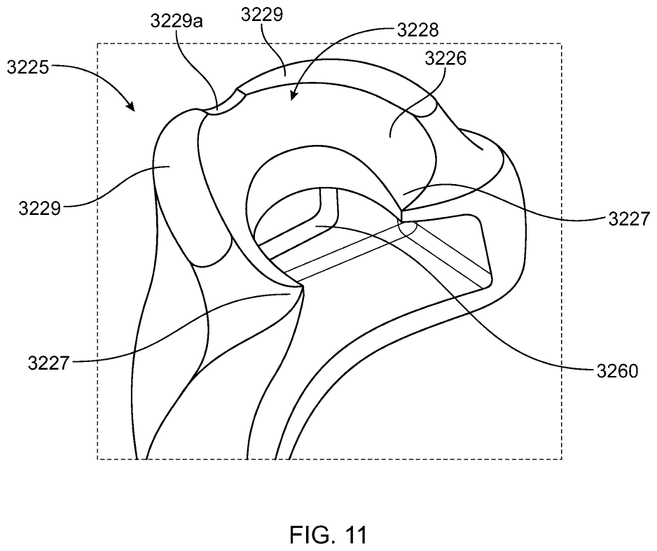

FIG. 11 is a posterosuperior view of an undercushion of the patient interface of FIG. 7.

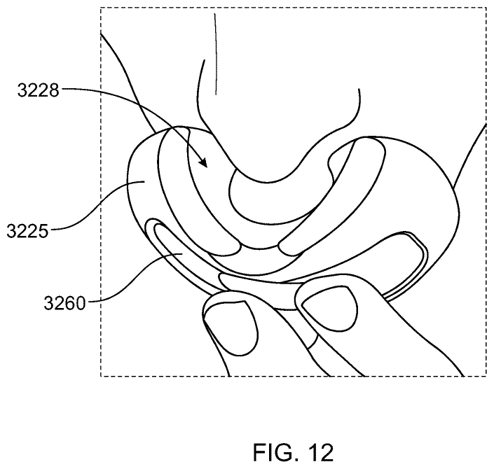

FIG. 12 is superior view of the undercushion of the patient interface of FIG. 7 in isolation and held close to an in-use position but held just off the surface of the patient's face to show how the relationship between the shape of the undercushion and the shape of the patient's face.

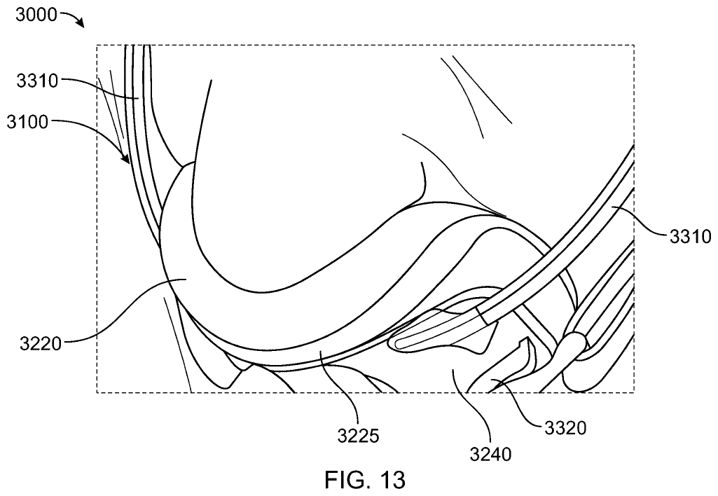

FIG. 13 is a superior detail view of the patient interface of FIG. 7 in an in-use position.

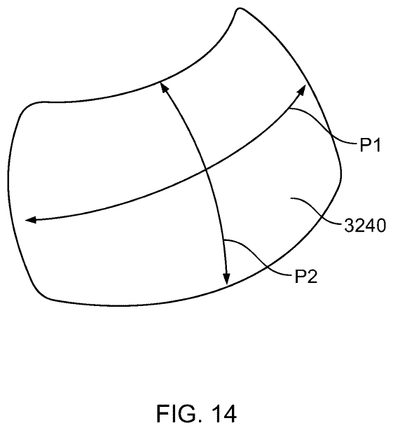

FIG. 14. is a diagrammatic view of a frame according to one form of the technology.

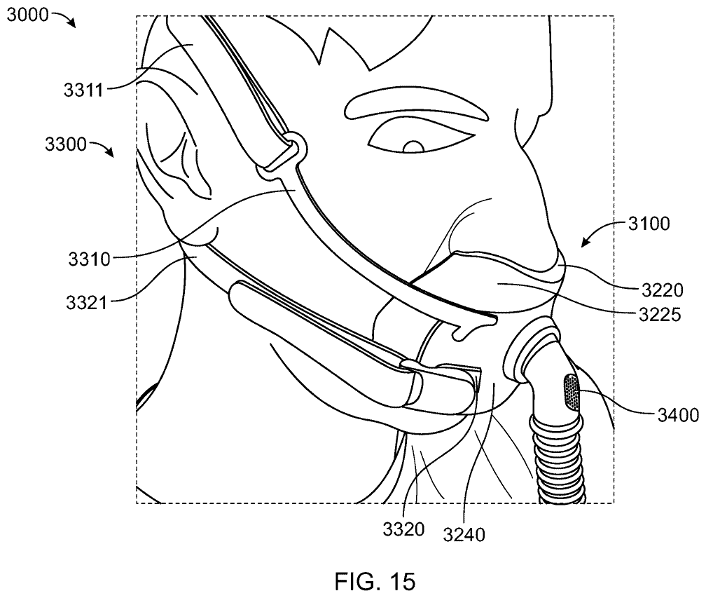

FIG. 15 is a side view of the patient interface of FIG. 7 worn by a patient and showing one side of the positioning and stabilising structure.

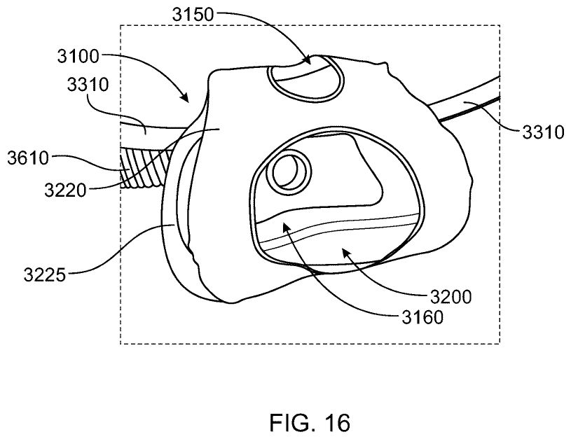

FIG. 16 is a view of a patient-contacting side of the patient interface of FIG. 7.

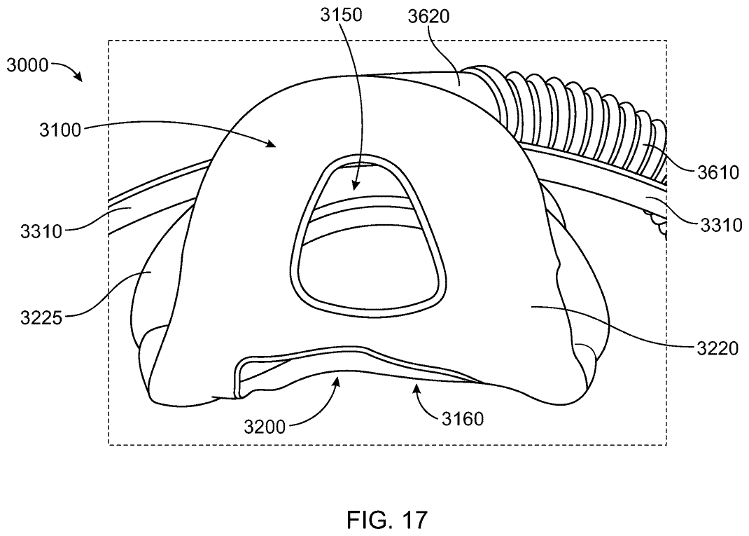

FIG. 17 is a detail view of a nasal portion of the patient interface of FIG. 7.

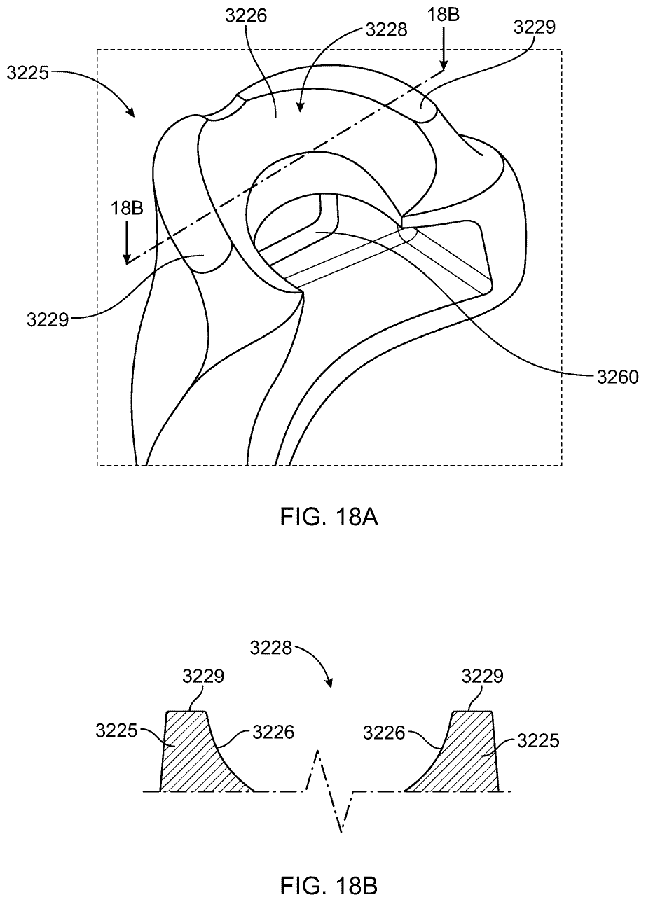

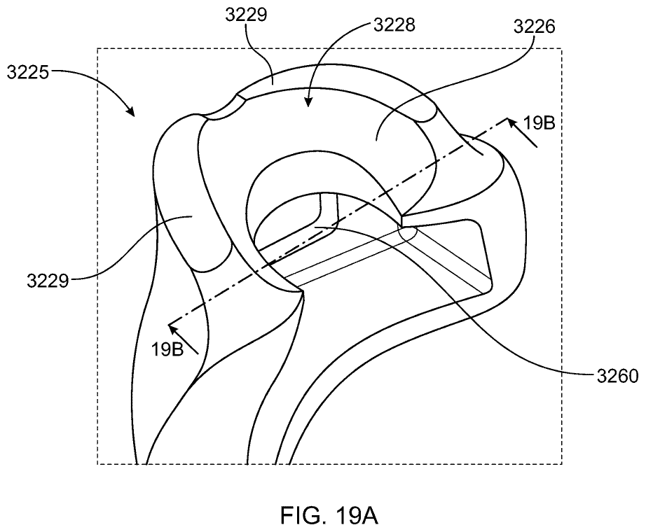

FIG. 18A is a perspective view of an undercushion according to one example of the present technology.

FIG. 18B is a cross section view through a nasal portion of the undercushion shown in FIG. 18A.

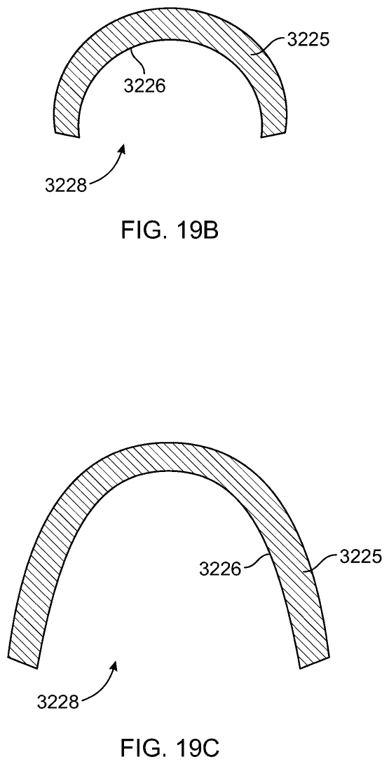

FIG. 19A is a perspective view of an undercushion according to one example of the present technology.

FIG. 19B is a cross section view through a nasal portion of the undercushion shown in FIG. 19B.

FIG. 19C is a cross section view at the same location as the cross section shown in FIG. 19B through a nasal portion of another undercushion according to an example of the present technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and, in some particular examples, a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH₂O with respect to ambient.

The patient interface 3000 in accordance with one form of the present technology is constructed and arranged to be able to provide a supply of air at a positive pressure of at least 10 cmH₂O with respect to ambient.

The patient interface 3000 in accordance with one form of the present technology is constructed and arranged to be able to provide a supply of air at a positive pressure of at least 20 cmH₂O with respect to ambient, for example up to 30 cmH2O or up to 40 cmH2O.

FIGS. 7-13 and 15-17 show various views of a patient interface 3000 according to an example of the present technology. In this example the patient interface 3000 comprises a frame 3240 partially forming a plenum chamber 3200 pressurisable to a therapeutic pressure of at least 6 cmH2O above ambient air pressure. The plenum chamber 3200 has at least one plenum chamber inlet port 3202 sized and structured to receive the flow of air at the therapeutic pressure for breathing by the patient.

The patient interface 3000 may further comprise at least one pair of headgear connectors connected to the frame 3240. In the illustrated example the patient interface 3000 comprises a pair of superior headgear connector portions 3310 and a pair of inferior headgear connector portions 3320. In other examples there may be only one pair of headgear connectors (e.g. a two-point headgear connection).

Also as shown in FIGS. 7-13 and 15-17, the patient interface 3000 further comprises a seal-forming structure 3100 connected to the frame 3240 and partially forming the plenum chamber 3200. The seal-forming structure 3100 is constructed and arranged to form a seal with a region of the patient's face surrounding an entrance to the patient's airways in use. The seal-forming structure 3100 has at least one hole therein such that the flow of air at the therapeutic pressure is delivered to an entrance to the patient's nares and the patient's mouth. The seal-forming structure 3100 is constructed and arranged to maintain said therapeutic pressure in the plenum chamber throughout the patient's respiratory cycle in use.

In some examples of the present technology, the seal-forming structure 3100 comprises an undercushion 3225. The seal-forming structure 3100 may further comprise a membrane portion 3220 connected to the undercushion 3225 and which is configured to inflate, in use, to form a seal to at least a pronasale region and nasal alae of the patient's nose. In this example the seal-forming structure 3100 is further configured to form a seal around the patient's mouth.

In the example shown in FIGS. 7-13 and 15-17 and in other examples, the undercushion 3225 and membrane 3220 may together form a cushion module. The cushion module may be combined with other components such as a frame 3240, a positioning and stabilising structure 3300 (e.g. headgear) and a connector 3620 and/or short tube 3610 for fluid connection to an air circuit, to form a patient interface 3000. The cushion module, and therefore the undercushion 3225 and membrane 3220, may be made available in multiple shape/size options, to fit a range of patient face shapes and sizes.

### 4.3.1 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200.

In the example shown in FIGS. 7-13 and 15-17, the plenum chamber 3200 is partially formed by the seal forming structure 3100 and partially formed by a frame 3240. The plenum chamber inlet port 3202 in this example is a single opening provided in the frame 3240. In particular, a short tube 3610 is connected to the frame 3240 and fluidly connected to the plenum chamber inlet port 3202 to convey a flow of air to the plenum chamber 3200. The patient interface 3000 comprises a connector 3620 connecting the short tube 3610 to the frame 3240.

### 4.3.2 Frame

In one form of the technology, the frame 3240 is flexible and may be formed from a flexible material. In some examples, the frame 3240 is formed from an elastomeric material, for example silicone or thermoplastic elastomer (TPE). In an example, the frame 3240 may be formed from a silicone having a 40 Shore A durometer hardness. Other suitable materials may be used. In some examples the frame may be formed from Mylar, for example. The frame 3240 in one example may be formed from silicone and may have a wall thickness of 2 mm. In other examples, the frame 3240 is substantially rigid and may be formed from a substantially rigid material, such as polycarbonate, or have a structure imparting substantial rigidity.

In some examples the frame 3240 comprises a non-zero negative first principle curvature and a second principle curvature which is less than the first principle curvature. In examples, the frame 3240 is curved such that it has a non-zero negative first principal curvature P1 and a substantially zero second principal curvature P2, as shown in FIG. 14.

In examples, the patient interface is configured such that the second principal curvature P2 is substantially parallel, in use, to a sagittal plane of the patient and may lie in the sagittal plane in use.

In some examples, the frame 3240 is formed substantially flat but is held in the curved configuration by the seal forming structure 3100. In other examples the frame 3240 may be inherently curved, for example it may be moulded or otherwise manufactured with an inherent curvature. In some examples the frame 3240 may be sufficiently flexible that despite being held in a curved configuration by an undercushion 3225, the frame 3240 substantially does not deform the undercushion 3225 (e.g. by reaction forces). In some examples the undercushion 3225 holds the frame 3240 in a curved shape across the user's face in the lateral-medial directions and allows for flexing of this curved shape, yet substantially prevents the cross section of the frame 3240 in the sagittal plane from flexing.

The patient interface 3000 may comprise at least one pair of headgear connectors connected to the frame 3240. The headgear connectors may be configured to connect to headgear straps of a positioning and stabilising structure 3300 of the patient interface 3000. As shown in FIGS. 7, 8 and 15 in particular, in some forms of the technology the frame 3240 is provided with a pair of superior headgear connector portions 3310 and a pair of inferior headgear connector portions 3320. In examples, the frame 3240 is also provided with an opening for connection (for example releasable connection) to an air delivery tube, in use. In examples the frame 3240 is provided with at least one vent 3400 for gas washout (e.g. to provide a continuous flow of gas from the plenum chamber 3200 to ambient throughout the patient's respiratory cycle in use). In the example shown in FIGS. 7 and 8, the frame 3240 comprises a connector 3620 providing a connection to a short tube 3610 having a connection port 3600 at the end thereof. The connector 3620 may be in the form of an elbow and/or may be configured to swivel with respect to the frame 3240. The vent 3400 in this example is provided to the connector 3620. The vent 3400 may be formed by a plurality of holes. As depicted in FIG. 15 (only one side visible), the superior headgear connector portions 3310 are connected to superior headgear straps 3311 and the inferior headgear connector portions 3320 are connected to inferior headgear straps 3321.

In examples in which the frame 3240 is relatively flexible, and has a substantially zero principal curvature substantially aligned with a sagittal plane, the frame 3240 may flex relatively easily such that the magnitude of the first principal curvature P1 can be varied when the patient interface 3000 is donned. For example, the magnitude of the first principal curvature may be relatively large (e.g. the frame 3240 may be more curved) when the patient interface 3000 is donned by a person having a relatively narrow face, but may be smaller (e.g. the frame 3240 may be relatively “flatter”) when the patient interface 3000 is donned by a person having a relatively broad face. The frame 3240 in such examples is configured to flex such that the first principle curvature has a larger magnitude when donned by a patient with a narrow face than when donned by a patient having a relatively wider face. In this way, the patient interface 3000 may be adaptable to fit patients having a range of different size faces. The use of silicone (e.g. 40 Shore A hardness silicone) to form the frame 3240 may provide for the above described flexing behaviour despite the frame 3240 comprising a C-shaped channel 3250 which due to its geometry and resultant effect on second moment of area, adds to the stiffness of the frame 3240. The channel 3250 in the frame will be described below in the context of the undercushion 3225.

As there is no preformed curvature in the frame 3240 in the sagittal plane (e.g. the principal curvature P2 described above), the frame 3240 may be configured to flex less readily in the sagittal plane (e.g. changing the second principal curvature P2) than in a horizontal plane (e.g. changing the first principal curvature P1). Furthermore, the frame 3240 is shorter in the superior-inferior directions than in the medial-lateral directions, which further makes the frame 3240 less likely to flex in the sagittal plane. In some examples the frame 3240 may comprise a small curvature in the sagittal plane (e.g. the principle curvature P2), either by being formed with a small curvature or by being held in a curved shape by the undercushion 3225.

### 4.3.3 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface 3000 was placed on the face, tension in the positioning and stabilising structure 3300 and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with some examples of the present technology may comprise a soft, flexible, resilient material such as silicone.

In other forms the seal forming structure 3100 comprises an undercushion 3225, which may be formed from foam, and a textile membrane portion 3220, as described further below.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head. However, examples of the technology may be suitable for a large range of heads, and so may be used by patients having a relatively large head and a relatively small head.

### 4.3.3.1 Undercushion

As shown in FIGS. 7-13 and 15-17, the seal forming structure 3100 comprises an undercushion 3225. The undercushion 3225 is formed from foam in the illustrated example but it is to be understood that the other materials and structures may also be used to form the undercushion, such as an elastomeric structure, an inflatable undercushion, a gel, a solid semi-rigid material, e.g. silicone or TPE. The undercushion 3225 is shaped to extend around the patient's mouth and be positioned proximate to an inferior periphery of the patient's nose in use. Examples of patient interfaces 3000 made in accordance with the present invention may be referred to as ultra-compact full face masks.

Attached to the undercushion 3225 is the membrane portion 3220 configured to contact the patient's face in use to form a seal around the nose and/or mouth, as will be described below. The undercushion 3225 may hold the membrane portion 3220 in place and in shape to form a seal to the patient's face. In some examples the undercushion 3225 may press the membrane portion 3220 against the user's face around the user's mouth. In some examples the undercushion 3225 may support the membrane portion 3220 around the patient's nose so that the membrane portion 3220 engages the patient's nose but the undercushion 3225 remains substantially clear of the patient's nose, e.g. to avoid nasal occlusion, as will be described.

### 4.3.3.1.1 Connection to Frame

The undercushion 3225 is connected, preferably removably, to the frame 3240. As shown in particular in FIGS. 8 and 9, the frame 3240 comprises a channel 3250 around a periphery of the frame 3240. The undercushion 3225 may comprise a connection portion 3260 on a non-patient facing side of the undercushion 3225, and the connection portion 3260 may be configured to be received in the channel 3250 of the frame 3240 to releasably connect the seal-forming structure 3100 to the frame 3240. When the patient wishes to separate the seal forming structure 3100 from the frame 3240 (for example to wash the seal forming structure 3100 or to replace it), the undercushion 3225 can be peeled away from the frame 3240. The connection portion 3260 of the undercushion 3225 may be a portion of the undercushion 3225 correspondingly shaped to the channel 3250 in the frame 3240. This manner of connection between the frame 3240 and undercushion 3225 may advantageously provide for easy attachment and disconnection of the frame 3240 and seal-forming structure 3100.

The channel 3250 may be defined by an anterior vertical wall, a horizontal wall and a posterior vertical wall, in some examples. It is to be understood that the channel 3250 may not necessarily be symmetrical in cross section. For example, in such a channel, the anterior vertical wall and posterior vertical wall need not be the same length. In some examples, the anterior vertical wall of the frame 3240 may be longer than the posterior vertical wall. This arrangement may result in an anterior wall of the frame 3240 covering more of an anterior external surface of the undercushion 3225 than a posterior wall covering an internal surface of the undercushion 3225.

In one example, the frame 3240 may be formed from silicone having a wall thickness of 2 mm defining an anterior wall of the frame 3240. The channel 3250 may be formed and defined by three walls of the frame, each having a wall thickness of 2 mm. The internal width of the channel 3250 may be 4 mm. The frame 3240 may have an overall section thickness of approximately 8 mm (comprised of the 2 mm anterior wall, the 4 mm internal width of the channel 3250 and the 2 mm posterior wall). In such an example the connection portion 3260 of the undercushion 3225 may have a thickness of substantially 5 mm. The foam (or other material, in other examples) forming the connection portion 3260 may compress when fitted to the 4 mm channel 3250, forming a good seal. More generally, the channel 3250 may be smaller than the connection portion 3260 of the undercushion 3225 provide for an interference fit for an airtight seal.

The connection between the undercushion 3225 and the frame 3240 is substantially airtight. That is, a seal is formed between the undercushion 3225 and the frame 3240 by the engagement between the channel 3250 and the undercushion 3225. The channel 3250 and the undercushion 3225 engage to form a seal. The frame 3240 may fit tightly to the undercushion 3225, which may advantageously create a good air seal, prevent blowout of the foam or other material forming the undercushion 3225 and transfer headgear forces to the undercushion 3225.

In the example shown in FIGS. 7-13 and 15-17, the channel 3250 extends around an entire periphery of the frame 3240. The channel 3250 in this example has a C-shaped cross section although in other examples the channel 3250 may comprise a different cross section. More generally, the channel 3250 may form a female connection portion corresponding to a male connection portion of the undercushion 3225. The channel 3250 in the example shown in FIGS. 7-13 and 15-17 opens outwardly with respect to the frame and the connection portion 3260 of the undercushion 3225 comprises an opening having a periphery facing inwardly with respect to the opening, the periphery being configured to fit within the channel 3250. The connection portion 3260 of the undercushion 3225 is in this example configured to be press fit into the channel 3250 by the user. In an alternative example a retention device such as a clip may be present to secure the undercushion 3225 in a channel 3250 of the frame 3240.

In comparison to a patient interface 3000 having an undercushion with a channel into which an edge of a frame is received, the channel 3250 being provided to the frame 3240 may advantageously provide for a robust and durable connection between frame 3240 and undercushion 3225. If the channel 3250 was formed in the undercushion 3225 it may make the undercushion 3225 more prone to tearing than otherwise, or may limit the expected service life of the undercushion 3225 more so than in the above described arrangement.

However, in some alternative examples the undercushion 3225 is provided with a female connection portion (e.g. a channel 3250) and the frame 3240 is provided with a male portion (e.g. a peripheral edge of the frame 3240) configured to be received in the undercushion 3225, for example if the undercushion 3225 is made from a sufficiently durable foam, has appropriate geometry (e.g. thickness) and/or has a channel formed by a more durable material.

In examples the height, width and or depth of the undercushion may vary between the frame and the textile membrane portion 3220. That is, the undercushion 3225 may not have a constant cross-section.

### 4.3.3.1.2 Nasal Recess

In one form of the technology shown in particular in FIGS. 11 and 12 the undercushion 3225 may comprise a nasal portion configured to be positioned proximate the inferior periphery of the patient's nose in use, the undercushion 3225 comprising a nasal recess 3228 in the nasal portion. The nasal recess 3228 may be shaped to receive the patient's nose, and may do so without engaging the user's nose or at least without applying an occluding force to the patient's nose. In some examples the nasal recess 3228 provides clearance between the undercushion 3225 and the inferior periphery of the patient's nose. The nasal recess 3228 may be configured to prevent the seal-forming structure 3100 from occluding the patient's nose, for example when the headgear is tightened. The membrane portion 3220 may be supported by the undercushion 3225 at a periphery of the nasal recess 3228. For example, the membrane portion 3220 may be attached to the undercushion 3225 at or proximate a periphery of the nasal recess 3228.

In some examples the nasal recess 3228 may space the undercushion 3225 from the patient's nose at least when the patient initially dons the patient interface 3000 prior to sleeping, so as to not press the membrane portion 3220 against the patient's nose, or at least not press the membrane portion 3220 against the nasal ala. The nasal recess 3228 may prevent the undercushion 3225 from engaging the patient's nose or at least prevent the undercushion 3225 from occluding the patients nose (e.g. by not engaging it with sufficient force to close or partially close the nostrils). The nasal recess 3228 may prevent inadvertent occlusion of the patient's nose which may otherwise occur if the undercushion 3225 was shaped to engage the nose together with the membrane portion 3220 (e.g. by pressing the membrane portion 3220 against the nasal alae). It is to be understood that on some patients with large noses the undercushion 3225 may nevertheless have a small amount of engagement with portions of a user's nose, but due to the nasal recess 3228 may not occlude the user's nose. For example, the nasal recess 3228 may be configured to avoid engaging both lateral sides of the patient's nose simultaneously. In some examples the nasal recess 3228 may be structured to provide substantially no medially-directed forces on the patient's nasal alae in use. In some forms, the nasal recess 3228 is structured and arranged such that the undercushion 3225 applies substantially no force on the patient's nose in use.

The undercushion 3225 and frame 3240 may be constructed such that in use they are positioned very close to the patient's face in use to allow for engagement between the undercushion 3225 with the patient's cheeks and mouth region. The nasal recess 3228 may allow for the undercushion 3225 to be urged against the patient's cheeks and mouth region without also excessively urging the patients nasal alae closed, which could occlude the patient's nasal airways.

The nasal recess 3228 (e.g. the portion of the undercushion 3225 forming the nasal recess 3228) in this example comprises a shape corresponding to the inferior periphery of the nose (e.g. the inferior shape of the pronasale portion and nasal alae) as shown in FIGS. 11 and 12. The corresponding shape, combined with only a small spacing between the nasal recess 3228 and patient's nose in use may provide for a low-profile patient interface 3000. As shown in FIG. 11 for example, the nasal recess 3228 may comprise an inwardly-facing wall 3226. The inwardly-facing wall 3226 may face towards the patient's nose and may also be described as a nasal-facing wall. For example, the inwardly-facing wall 3226 may be structured to face and at least partially surround the inferior periphery of the patient's nose in use. The inwardly-facing wall 3226 may be one or more of substantially chamfered, frustoconical, bowl-shaped, frustospherical, parabolic, among other possible shapes. The inwardly-facing wall 3226 may be concave, in some examples concave in two orthogonal planes. For example, the inwardly-facing wall 3226 may comprise a concave shape when viewed in a vertical cross-section (e.g. a cross-section parallel to the sagittal or coronal plane) and also when viewed in a horizontal cross-section (e.g. a cross-section parallel to the Frankfort horizontal). In other examples the inwardly-facing wall 3226 may not be concave in a vertical cross section, for example if the inwardly-facing wall 3226 is chamfered or otherwise appears as a straight line in vertical cross section. In some examples the inwardly-facing wall 3226 may appear as a straight line in vertical cross section in one location (e.g. proximate the pronasale) but may appear as a curved line in another location (e.g. on a lateral side of the patient's nasal ala), or vice versa.

FIG. 18B shows a cross-section view of the nasal portion of the undercushion 3225 in a cross-sectional plane oriented parallel to the coronal plane as indicated in FIG. 18A. As shown in FIG. 18B, the inwardly-facing wall 3226 comprises a pair of lateral portions on either lateral side of the nasal recess 3228. The lateral portions may face medially. These are positioned in use on either lateral side of the user's nose in use. Also evident from FIG. 18B is that the inwardly-facing wall 3226 comprises a positive curvature, making it concave. The lateral portions of the inwardly-facing wall 3226 may face partially medially and partially superiorly, as shown in FIG. 18B. The inwardly-facing wall 3226 may comprise the same or a similar shape at an anterior portion of the inwardly-facing wall 3226, for example at a location proximate the patient's pronasale. The anterior portion of the inwardly-facing wall 3226 may face posteriorly and may be positioned anterior and/or inferior to the patient's pronasale. The anterior portion of the inwardly-facing wall 3226 may face partially posteriorly and partially superiorly. Similarly to the lateral portions of the inwardly-facing wall 3226, the anterior portion of the inwardly-facing wall 3226 may have a positive curvature and may be concave.

FIG. 19B shows a cross-section view of the nasal portion of the undercushion 3225 in a horizontal cross-sectional plane (e.g. a horizontal plane parallel to the Frankfort horizontal plane) as indicated in FIG. 19A. As shown in FIG. 19B, the inwardly-facing wall 3226 in this example is concave in a horizontal plane. The concavity in a horizontal plane results in the nasal recess 3228 surrounding the patient's nose. This shape is depicted schematically in FIG. 19B as a semi-circle and it is to be understood that, in various examples, the shape of the nasal recess 3228 and the inwardly-facing wall 3226 thereof in a horizontal plane may be semi-circular or may be a non-circular concave shape, such as a shape closer to a triangle with a rounded anterior corner, lateral sides and no base. In some examples, the nasal recess 3228 may generally have a shape in a horizontal plane that follows a general shape of the patient's nose, for example the inferior periphery of the patient's nose. FIG. 19C shows schematically the shape of the nasal recess in another example. In this example the inwardly-facing wall 3226 comprises an concave anterior portion facing posteriorly and two lateral portions. The lateral portions may face predominantly medially and to a lesser extent posteriorly. Such a shape may more closely follow the shape of a narrow and long nose. The shape shown in FIG. 19B may more closely follow the shape of a wide and short nose. In some examples the undercushion 3225 (or a cushion module comprising the undercushion 3225) are provided in a range of sizes and/or shapes, the shape of the nasal recess 3228 and inwardly-facing wall 3226 thereof differing the range of cushion module options so that each patient can select the cushion module having the best fitting undercushion 3225 for their nose and face.

The nasal recess 3228 in some examples surrounds substantially all of the laterally and anteriorly facing portions of the inferior periphery of the patient's nose in use. The inwardly-facing wall 3226 may extend from at or proximate one of the patient's cheeks around the patient's nose to at or proximate the other of the patient's cheeks.

With reference to FIGS. 11 and 12, the undercushion 3225 in this particular example further comprises a pair of posterior support portions 3227 each positioned on a respective lateral side of the nasal recess 3228 and configured to engage the patient's face medially of and proximate to the nasolabial sulci of the patient's face. These posterior support portions 3227 may engage the patient's face at locations inferior to the nasal alae on either lateral side of the patient's lip superior, or may engage the patient's face at locations aligned vertically with the patient's nasal alae between the nasal alae and the nasolabial sulci, for example. In some examples, the posterior support portions 3227 may engage the patient's face in an area that extends vertically from alongside the nasal alae to alongside the patient's lip superior. The posterior support portions 3227 may engage the face to support the patient interface 3000 in position and may also advantageously encourage the membrane portion 3220 to form a good seal proximate the inferior corners of the patient's nose, which may be more difficult to seal against on a wide range of patients in comparison to other places such as the cheeks. As shown in FIG. 11 for example, the inwardly-facing wall 3226 may extend from a first one of the posterior support portions 3227 around the patient's nose to the other one of the posterior support portions 3227.

The posterior support portions 3227 in the example shown in FIG. 11 also each include a region of the inwardly-facing wall 3226, although this may be a relatively small region in comparison to the overall size of the inwardly-facing wall 3226. The posterior support portions 3227 are in this example each shaped to protrude at least partially medially into concavities formed on the patient's face on either lateral side of the nasal alae, for example between the nasal alae and adjacent portions of the patient's face. For example, a posterior-facing surface or portion of each posterior support portion 3227 may engage the patient's face in a region between the nasolabial sulcus and nasal ala. At the same time, a portion of the inwardly facing surface 3226 forming part of or located proximate to each of the posterior support portions 3227 may engage a respective one of the patient's nasal alae. The posterior support portions 3227 may “dig in” to the concavities on either side of the patient's nose to assist in achieving a good seal in those regions. The posterior support portions 3227 may engage only a rearmost portion of the nasal ala. The posterior support portions 3227 may contact the nasal ala for the purpose of encouraging sealing of the concavity at either lateral side of the inferior periphery of the patient's nose. The undercushion 3225 is still configured to avoid exerting medially-directed forces on the patient's nose which may tend to occlude the nasal airways.

In the example shown in FIGS. 7-13 the membrane portion 3220 is attached to the undercushion 3225 around the periphery of the nasal recess 3228. As shown in FIGS. 11 and 12 in particular, the undercushion 3225 comprises a superiorly-facing surface 3229 located adjacent to and provided around the periphery of the nasal recess 3228, the membrane portion 3220 being attached to the superiorly-facing surface 3229 of the undercushion 3225. The membrane portion 3220 may be glued, bonded or otherwise attached to the superiorly-facing surface 3229. The superiorly-facing surface 3229 may be flat, in some examples. In other examples the superiorly-facing surface 3229 may have a small curvature in cross section but not to an extent that the membrane portion 3220 cannot robustly attach to the superiorly-facing surface 3229. In other examples the membrane portion 3220 is attached to an attachment surface of the nasal portion which may or may not be superiorly-facing. This portion defining the nasal recess 3228 may have a consistent wall cross section around the nasal recess 3228 to avoid weak spots in some examples. That said, in some examples the nasal portion of the undercushion 3225 may comprise a medial recess 3229a in the superiorly-facing surface 3229. The medial recess 3229a may be configured to provide clearance for the pronasale and/or reduce pressure on the pronasale in the event there is inadvertent engagement between the undercushion 3225 and the pronasale. While the nasal recess 3228 and medial recess 3229a are intended to provide clearance for the nose, and pronasale thereof, respectively, it is to be understood that for some patients there may nevertheless be some engagement between the undercushion 3225 and nose in one or more locations. However, the nasal recess 3228 and medial recess 3229a are still to be understood to be configured (e.g. structured, shaped, arranged and/or positioned) to provide clearance as they are generally shaped to avoid contact with the nose and pronasale, respectively, and result in less contact than would otherwise occur.

In examples the undercushion 3225 formed from a foam material is made from a polyurethane, either a thermoplastic or thermoset polyurethane, or from a thermoplastic elastomer (e.g. a soft TPE having similar behaviour to a foam). Foam having the following properties may be suitable:
Force Deflection: 95+/−20N @40% (Test spec: AS 2282.8 Method A)
Density: 54.5+/−2.5 kg/m3 (Test spec: AS 2282.3)
Air Permeability: <1.5 l/min @ 20 cm H2O (R630-602 Permeability Test (Annular) Procedure).

In some examples, foam having a lower density than the above may be used to form the undercushion 3225. In some examples the undercushion 3225 may be formed from a low density foam but may have a sufficiently thick base or body to it that it is able to sufficiently support the textile membrane 3220 and support itself, especially in the nose region.

In other examples any of a polyethylene foam, a silicone foam or an EVA foam may be used, among other examples.

In some examples the foam undercushion 3225 may be formed by moulding, e.g. injection moulding.

In examples the foam may be substantially water impermeable so that the mask can dry adequately before use at night when washed earlier in the day.

In examples the undercushion material is soft enough to cause substantially no discomfort when engaging the face, but is firm enough to retain the frame and hold it in a sealed manner when the patient interface is worn.

In examples the foam from which the undercushion 3225 is made is substantially air impermeable, for example, the foam may be a skinned foam.

In use, the undercushion 3225 supports the membrane portion 3220.

### 4.3.3.2 Membrane Portion

As discussed above, the patient interface 3000 may comprise a membrane portion 3220 which engages the patient's face. The membrane portion 3220 may be at least partially formed from a textile material, from an elastomer such as silicone or TPE, or from another suitable material, in various examples. Referring next to FIGS. 10 and 13 in particular, in one form, the seal-forming structure includes a textile membrane portion 3220 which functions as a pressure assisted sealing mechanism. In use, the textile membrane portion 3220 can readily respond to a system positive pressure in the interior of the plenum chamber 3200 acting on its interior side to urge it into tight sealing engagement with the face. The pressure assisted mechanism may act in conjunction with elastic tension in the positioning and stabilising structure 3300. In other examples the membrane portion 3220 may be formed from silicone, such as a silicone membrane having a thickness of 0.2-0.4 mm, or 0.25-0.3 mm, for example.

As shown in FIG. 16, in some examples, the membrane portion 3220 is connected at its outer periphery to a patient facing side of the undercushion 3225, such that the membrane is substantially taut when not in use. The membrane portion 3220 may be bonded to the undercushion 3225, for example. In examples, the only connection between the textile membrane portion 3220 and the undercushion 3225 is at the outer periphery of the textile membrane portion 3220. When subject to treatment pressure inside the plenum chamber 3200, the textile membrane portion 3220 may “balloon” under the pressure. This may cause the textile membrane portion 3220 to conform closely to the shape of the patient's face. In one form, the membrane portion 3220 may be configured to form a seal to at least a pronasale region and to the nasal alae of the patient's nose and may also seal to the patient's lip superior. The seal-forming structure 3100, for example a portion of the membrane portion 3220 or another portion, may be further configured to form a seal around the patient's mouth.

In some examples, the membrane portion 3220 is formed from a single sheet of textile. The membrane portion 3220 may comprise a first hole 3150 through which air can flow from the plenum chamber 3200 to both the patient's nares, and a second hole 3160 through which air can flow from the plenum chamber 3200 to the patient's mouth. The membrane portion 3220 may extend between the first and second holes 3150, 3160. The first hole 3150 may be substantially triangular with rounded sides and corners, for example, as shown in FIG. 17. The posterior side of the first hole 3150 may be wider than the anterior side, to correspond to the general shape of an inferior periphery of the patient's nose.

In other examples (not shown), there may be a single hole in the membrane portion 3220 for both the patient's mouth and nares. In examples the membrane portion 3220 may effectively have only a single hole, but opposed lateral sides of the membrane portion 3220 at the edge of the hole may be connected by a bridging portion, for example a textile bridging portion, which may be located directly across the patient's lip superior.

The membrane portion 3220 may be attached to the undercushion 3225 around a periphery of the nasal recess 3228 described above. The membrane portion 3220 may be attached to the undercushion 3225 across the nasal recess 3228 such that the membrane portion 3220 is taut when the patient interface 3000 is not in use. This taut configuration may mean that when the membrane portion 3220 is brought into contact with the patient's nose and the patient's nose is pressed into the nasal recess 3228, the first hole 3150 in the membrane portion 3220 will locate directly under the nose and will stretch tight around the patient's nares in preparation for a pressurised seal in use. The application of pressurised air in the plenum chamber 3200 will then push against the membrane portion 3220 and apply additional sealing force around the first hole 3150.

As shown in FIG. 13 in particular, the patient's nose presses into the membrane portion 3220 above the nasal recess 3228 and the membrane portion 3220 “cups” the underside of the nose forming a seal to the edges of the nose. The inferior periphery of the patient's nose may in use be located within the nasal recess 3228 but spaced from the undercushion 3225 around the periphery of the nasal recess 3228. The membrane portion 3220 may bridge the gap between the patient's nose and the undercushion 3225 around the periphery of the nasal recess 3228. The inwardly-facing wall 3226 may be spaced from the patient's nose in use or, if the inwardly-facing wall 3226 does inadvertently engage the patient's nose in some patients, it may do so with minimal or substantially no force.

In some examples, the membrane portion 3220 may be configured to be stretched in use by the patient's nose proximate the first hole 3150. The membrane portion 3220 may be configured to stretch around the base of the nares when the nose is placed onto the membrane portion 3220 and pressed down into the nasal recess 3228. The stretching of the first hole 3150 may enable the membrane portion 3220 to lie flat and tight against the underside of the patient's nose. The stretching of the first hole 3150 may also relieve tension in the membrane portion 3220 which may prevent or provide only a low likelihood that the portions of the undercushion 3225 defining the sides of the nasal recess 3228 get pulled into the nasal recess 3228 which could cause nasal occlusion.

The textile membrane portion 3220 may be coated with an air impermeable coating, for example a silicone coating. In one example the silicone coating has a thickness within the range of 0.02 mm-0.05 mm (or within the range of 0.03 mm-0.05 mm) and the textile membrane portion 3220, including the coating, is substantially 0.3 mm thick. The coating may be on a non-patient contacting side of the textile membrane portion 3220.

The textile membrane portion 3220 may comprise a knitted fabric. The membrane portion 3220 may be highly flexible so as to readily conform to the shape of the patient's face. In examples the textile may comprise polyamide (e.g. nylon), polyester and/or elastane (e.g. spandex).

In one example a textile with the following properties may be used:
Single jersey knit;
Content: polyamide 80%, elastane 20%;
Density: 136 course per inch, 75 wale per inch;
Weight: 105 g/m²; and
Elongation at 1.5 kg: Length 80-108%, Width 93-126%.

In examples the frame 3240 and/or undercushion 3225 are shaped such that the patient facing side of the textile membrane portion 3220 lies on a continuously convex curve when viewed in cross-section through a sagittal plane (for example a central sagittal plane). In some examples it is solely the undercushion 3225 that holds the shape of the membrane portion 3220 due to high flexibility in the frame 3240.

For example, the textile membrane portion 3220 may have no secondary geometrical features which could create creases. This allows the membrane to be held in a configuration in which no creases are created when the patient interface 3000 is not in use. When the patient interface 3000 is applied to the face, the membrane portion 3220 flexes and forms to the face, particularly around the peripheries of the nose and mouth holes, as these may be the first points of engagement.

The highly compliant textile membrane portion 3220 that is activated with pressure may advantageously allow for a robust seal. The treatment pressure when applied to the compliant textile membrane portion 3220 may allow it to form readily to varying face shapes. The compliant nature of the textile membrane and/or the large area of the membrane in contact with the patient's face may assist in reducing areas of discomfort between the seal forming structure 3100 and the patient's face, as may be present with less compliant seal forming structures of the prior art. In examples the textile may be of a fine knit. The surface of the textile may be relatively slippery in order to allow the patient to more easily position the interface when donning the interface, and to ensure that the fabric does not remain creased against the patient's skin.

### 4.3.4 Positioning and Stabilising Structure

The seal-forming structure 3100 of the patient interface 3000 of the present technology may be held in sealing position in use by a positioning and stabilising structure 3300.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

### 4.3.5 Connectors for Positioning and Stabilising Structure

As best seen in FIGS. 7 and 8, in examples, connectors for connecting the positioning and stabilising structure 3300 to the patent interface 3000 are provided to the frame 3240. In examples the frame 3240 is provided with a pair of superior headgear connector portions 3310 and a pair of inferior headgear connector portions 3320.

Each of the superior headgear connector portions 3310 may comprise a curved arm. In the example shown in FIGS. 7 and 8 the superior headgear connector portions 3310 take the form of rigidised arms, each arm provided with a strap engagement means (e.g. a loop or a slot) for engaging a headgear strap at or proximate the end thereof. The rigidised arms may extend laterally from the frame 3240 and then curve to extend posteriorly in use. The arms may also curve towards a superior direction, in use.

In the example shown in FIGS. 7 and 8 the inferior headgear connector portions 3320 comprise magnetic connectors, which may engage complementary connectors attached to inferior headgear straps.

As depicted in FIG. 15 (only one side visible), the superior headgear connector portions 3310 are connected to superior headgear straps 3311 and the inferior headgear connector portions 3320 are connected to inferior headgear straps 3321.

It is to be understood that any suitable positioning and stabilising structure 3300 may be provided to patient interfaces 3000 according to examples of the present technology, and any suitable corresponding headgear connectors may be provided to the frame 3240 or cushion module.

### 4.3.6 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO₂ by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel. In the example shown in FIGS. 7-13 and 15-17, the vent 3400 of the patient interface 3000 is located in the connector 3620. In other examples it may be located in the frame 3240 or even in the undercushion 3225.

### 4.3.7 Decoupling Structure(s)

In one form the patient interface 3000 includes at least one decoupling structure, for example, a swivel or a ball and socket.

As described above, the frame 3240 is connected to a short tube 3610 by a connector 3620 in the example shown in FIGS. 7-13 and 15-17. The connector 3620 in this example is in the form of an elbow. In other examples it may be a straight connector. The connector 3620 provides a ball and socket joint connection to the frame 3240 in this example to provide for multiple axes of rotation and, in other examples, may be connected to the frame so as to rotate around only a single axis (e.g. an axis coaxial with a hole in the frame 3240). In such an example the connector 3620 may comprise an additional portion which rotates independently of the portion that rotates within the frame 3240, to provide an additional axis of rotation available to the connection between the short tube 3610 and the frame 3240. The connector 3620 may be removably attached to the frame 3240. The connector 3620, by being able to rotate with respect to the frame 3240 about one or more axes, forms a decoupling structure.

The short tube 3610 in the example shown in FIGS. 7-13 and 15-17 also forms a decoupling structure as it decouples movement of an air circuit 4170, to which it is attached, from the frame 3420, at least partially mitigating the effect of tube drag in use. Furthermore, in some examples the connection port 3600 may comprise a swivel connection to the air circuit, providing for additional or alternative decoupling of the air circuit 4170 from the frame 3420.

### 4.3.8 Connection Port

Connection port 3600 allows for connection to the air circuit 4170. In the example shown in FIGS. 7-13 and 15-17 the connection port 3600 is at a distal end of the short tube 3610. In other example the connection port 3600 may be attached to the frame 3240 without a short tube 3610. In such an example the connection port 3600 may be provided by an elbow fluidly connected to the frame 3240 and configured to fluidly connect to the air circuit 4170.

### 4.3.9 Forehead Support

In one form, the patient interface 3000 includes a forehead support 3700.

### 4.3.10 Anti-Asphyxia Valve

In one form, the patient interface 3000 includes an anti-asphyxia valve.

### 4.3.11 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.4 RPT Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH₂O, or at least 10 cmH₂O, or at least 20 cmH₂O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., one or more air filters 4110 such as an inlet air filter 4112 and/or outlet air filter 4114, an inlet muffler 4122, a pressure generator 4140 capable of supplying air at positive pressure (e.g., a blower 4142 comprising a motor 4144), one or more mufflers 4120 such as an outlet muffler 4124 and one or more transducers 4270, such as pressure sensors and flow rate sensors.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016. An anti spillback valve 4160 may be provided between the pneumatic block 4020 and the humidifier 5000.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components may be mounted on a single Printed Circuit Board Assembly (PCBA) 4202. In an alternative form, the RPT device 4000 may include more than one PCBA 4202.

### 4.4.1 RPT Device Algorithms

As mentioned above, in some forms of the present technology, the central controller 4230 may be configured to implement one or more algorithms expressed as computer programs stored in a non-transitory computer readable storage medium, such as memory. The algorithms are generally grouped into groups referred to as modules.

In other forms of the present technology, some portion or all of the algorithms may be implemented by a controller of an external device such as the local external device or the remote external device. In such forms, data representing the input signals and/or intermediate algorithm outputs necessary for the portion of the algorithms to be executed at the external device may be communicated to the external device via the local external communication network or the remote external communication network. In such forms, the portion of the algorithms to be executed at the external device may be expressed as computer programs stored in a non-transitory computer readable storage medium accessible to the controller of the external device. Such programs configure the controller of the external device to execute the portion of the algorithms.

In such forms, the therapy parameters generated by the external device via the therapy engine module (if such forms part of the portion of the algorithms executed by the external device) may be communicated to the central controller to be passed to the therapy control module.

### 4.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 4.5.1 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 4.6 Humidifier

### 4.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240. The reservoir 5110 comprises a conductive portion 5120 configured to allow efficient transfer of heat from the heating element 5240 to the volume of liquid in the reservoir 5110. The reservoir 5110 may comprise a water level indicator 5150.

In some arrangements, the humidifier reservoir dock 5130 may comprise a locking feature such as a locking lever 5135 configured to retain the reservoir 5110 in the humidifier reservoir dock 5130

### 4.7 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 4.8 Respiratory Therapy Modes

Various respiratory therapy modes may be implemented by the disclosed respiratory therapy system including CPAP therapy, Bi-level therapy and/or High Flow therapy.

### 4.9 Glossary

For the purposes of the present technology disclosure, in certain forms of the present technology, one or more of the following definitions may apply. In other forms of the present technology, alternative definitions may apply.

### 4.9.1 General

Air: In certain forms of the present technology, air may be taken to mean atmospheric air, and in other forms of the present technology air may be taken to mean some other combination of breathable gases, e.g. atmospheric air enriched with oxygen.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m2=1 millibar ˜ 0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy (RPT): The application of a supply of air to an entrance to the airways at a treatment pressure that is typically positive with respect to atmosphere.

Ventilator: A mechanical device that provides pressure support to a patient to perform some or all of the work of breathing.

### 4.9.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 4.9.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.9.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

(inhalation) Time (Ti): The duration of the inspiratory portion of the respiratory flow rate waveform.

(exhalation) Time (Te): The duration of the expiratory portion of the respiratory flow rate waveform.

(total) Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.9.3 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the respiratory portion of the breathing cycle by the patient's efforts.

### 4.9.4 Anatomy

### 4.9.4.1 Anatomy of the Face

Ala: the external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius):

Lip, upper (labrale superius):

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### 4.9.4.2 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 4.9.4.3 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.9.5 Patient Interface

Anti-asphyxia valve (AAV): The component or sub-assembly of a mask system that, by opening to atmosphere in a failsafe manner, reduces the risk of excessive CO₂ rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.9.6 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 4.9.6.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 4.9.6.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical-topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 4.9.6.3 Space Curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 3O and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S

With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 4.9.6.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 4.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1.-54. (canceled)

55. A patient interface comprising:
a frame formed from a flexible material and partially forming a plenum chamber pressurisable to a therapeutic pressure of at least 6 cmH2O above ambient air pressure, the frame comprising a channel around a periphery of the frame, the plenum chamber having at least one plenum chamber inlet port sized and structured to receive a flow of air at the therapeutic pressure for breathing by the patient;
at least one pair of headgear connector portions connected to the frame;
a seal-forming structure releasably connectable to the frame and partially forming the plenum chamber, the seal-forming structure being constructed and arranged to form a seal with a region of the patient's face surrounding an entrance to the patient's airways, the seal-forming structure having at least one hole therein such that the flow of air at the therapeutic pressure is delivered to an entrance to the patient's nares and the patient's mouth, the seal-forming structure constructed and arranged to maintain said therapeutic pressure in the plenum chamber throughout the patient's respiratory cycle in use,
wherein the seal forming structure comprises a foam undercushion and a membrane portion connected to the foam undercushion and configured to inflate, in use, to form a seal to at least a pronasale region and nasal alae of the patient's nose, the seal forming structure further configured to form a seal around the patient's mouth;
wherein the foam undercushion of the seal-forming structure comprises a connection portion on a non-patient facing side of the foam undercushion, the connection portion being configured to be received in the channel of the frame to releasably connect the seal-forming structure to the frame.

56. The patient interface of claim 55, wherein the frame is formed from an elastomeric material.

57. The patient interface of claim 55, wherein the channel extends around substantially an entire periphery of the frame.

58. The patient interface of claim 55, wherein the channel and the foam undercushion engage to form a seal.

59. The patient interface of claim 55, wherein the channel has a substantially C-shaped cross-section.

60. The patient interface of claim 55, wherein the channel opens outwardly with respect to the frame and the connection portion of the foam undercushion comprises an opening having a periphery facing inwardly with respect to the opening, the periphery of the opening being configured to fit within the channel.

61. The patient interface of claim 55, wherein the connection portion is configured to be press fit into the channel.

62. The patient interface of claim 55, wherein the foam undercushion is configured to hold the membrane portion substantially taut when the patient interface is not in use.

63. The patient interface of claim 55, wherein the membrane portion is attached to the foam undercushion around an outer periphery of the membrane portion.

64. The patient interface of claim 55, wherein the membrane portion is bonded to the foam undercushion.

65. The patient interface of claim 55, wherein the membrane portion comprises a first hole through which air can flow to both the patient's nares, in use.

66. The patient interface of claim 65, wherein the membrane portion is configured to be stretched by the patient's nose proximate the first hole in use.

67. The patient interface of claim 65, wherein the membrane portion comprises a second hole through which air can flow to the patient's mouth, in use.

68. The patient interface of claim 55, wherein the membrane portion is at least partially formed from a textile material.

69. The patient interface of claim 55, wherein the foam undercushion comprises a nasal portion configured to be positioned proximate an inferior periphery of the patient's nose in use, the foam undercushion comprising a nasal recess in the nasal portion configured to prevent the seal-forming structure from occluding the patient's nose.

70. The patient interface of claim 69, wherein the nasal recess comprises a shape corresponding to the inferior periphery of the patient's nose.

71. The patient interface of claim 69, wherein the membrane portion is attached to the foam undercushion around the periphery of the nasal recess.

72. The patient interface of claim 55, wherein the frame has a non-zero negative first principal curvature and a second principal curvature which is less than the first principal curvature.

73. The patient interface of claim 72, wherein the second principal curvature is substantially zero and substantially parallel, in use, to the patient's sagittal plane.

74. The patient interface of claim 72, wherein the frame flexes such that the first principal curvature has a larger magnitude when donned by a patient with a narrow face than when donned by a patient having a relatively wider face.
